(19) United States
(12) Patent Application Publication
(10) Pub. No.: US 2004/0127440 A1
(43) Pub. Date: Jul. 1, 2004

(54) Methods and compositions for control of bone formation via modulation of neuropeptide Y activity

(75) Inventors: Gerard Karsenty, Houston, TX (US); Patricia Ducy, Houston, TX (US); Michael Amling, Hamburg (DE)

Correspondence Address: ROPES & GRAY LLP, ONE INTERNATIONAL PLACE, BOSTON, MA, 02110-2624, US

(73) Assignee: BAYLOR COLLEGE OF MEDICINE

(21) Appl. No.: 10/465,910

(22) Filed: Jun. 17, 2003

Related U.S. Application Data
(63) Continuation of application No. 09/489,872, filed on Jan. 20, 2000, now abandoned.

(51) Int. Cl. A61K 48/00

(52) U.S. Cl. 514/44

(57) ABSTRACT

The invention relates to the method for treatment, diagnosis and prevention of bone disease and comprises methods including inhibiting or increasing neuropeptide Y synthesis, neuropeptide Y receptor synthesis, neuropeptide Y binding to the neuropeptide Y receptor, and neuropeptide Y receptor activity. The invention also relates to screening assays to identify compounds that modulate neuropeptide Y and/or neuropeptide Y receptor activity. The invention further relates to gene therapy methods utilizing neuropeptide Y and neuropeptide Y-related sequences for the treatment and prevention of bone disease.

This invention was made with government support under grant numbers NIH RO1 DE11290, NIH RO1 AR45548 and NIH RO1 AR43655, awarded by National Institute of Health. The government may have certain rights in the invention.

### 1 INTRODUCTION

The present invention relates to compositions and methods for the treatment, diagnosis and prevention of conditions, disorders or diseases involving bone, including, but not limited to, osteoporosis. The invention relates to modulation of the receptor signaling pathway for the polypeptide hormone neuropeptide Y (“NPY”). More particularly the present invention relates to the modulation of NPY synthesis, NPY-receptor synthesis, NPY binding to its receptor, and NPY signaling to bone cells.

The present invention also provides methods for the identification and prophylactic or therapeutic use of compounds in the treatment and diagnosis of conditions, disorders, or diseases involving bone. Additionally, methods are provided for the diagnostic monitoring of patients undergoing clinical evaluation for the treatment of conditions or disorders involving bone, for monitoring the efficacy of compounds in clinical trials and for identifying subjects who may be predisposed to such conditions, disorders, or diseases involving bone.

### 2 BACKGROUND OF THE INVENTION

The physiological process of bone remodeling allows constant renewal of bone through two well-defined sequential cellular processes. Karsenty, 1999, Genes and Development, 13:3037-3051. The initial event is resorption of preexisting bone by the osteoclasts, followed by de novo bone formation by the osteoblasts. These two processes in bone remodeling must maintain equilibrium of bone mass within narrow limits between the end of puberty and the arrest of gonadal function. The molecular mechanisms responsible for maintaining a constant bone mass are unknown, yet several lines of evidence suggest that this may be achieved, at least in part, through a complex endocrine regulation. For example, gonadal failure and the concomitant deficiency of the sex steroids stimulates the bone resorption process of bone remodeling and eventually leads to osteopenia (low bone mass) or osteoporosis (low bone mass and high susceptibility to fractures). Likewise, the recent identification of osteoprotegerin in serum and its functional characterization through a systemic route is another indication that secreted molecules affect osteoclastic bone resorption. Simonet et al., 1997, Cell, 89:309-319. This systemic control of bone resorption suggests that other circulating molecules, yet to be identified, could control bone formation via the osteoblasts. The identification of these hormones or growth factors, if they exist, is of paramount importance given the incidence and morbidity of diseases affecting bone remodeling.

One such disease is osteoporosis. Riggs et al., 1998, J. Bone Miner. Res., 13:763-773. Osteoporosis is the most common disorder affecting bone remodeling and the most prevalent disease in the Western hemisphere. At the physiopathological level, hallmarks of the disease are that bones are less dense and, thus, subject to fractures. In addition, the onset of osteoporosis in both sexes is intimately linked to arrest of gonadal function and is rarely observed in obese individuals. At the cellular level, it is characterized by a loss in equilibrium of bone remodeling favoring bone resorption over bone formation, which leads to bone fractures. At the molecular level, the pathogenesis of osteoporosis remains largely unknown.

### 3 SUMMARY OF THE INVENTION

An object of the present invention is the treatment, diagnosis and/or prevention of bone disease through manipulation of the NPY signaling pathway. Bone diseases which can be treated and/or prevented in accordance with the present invention include bone diseases characterized by a decreased bone mass relative to that of corresponding non-diseased bone, including, but not limited to osteoporosis, osteopenia and Paget's disease. Bone diseases which can be treated and/or prevented in accordance with the present invention also include bone diseases characterized by an increased bone mass relative to that of corresponding non-diseased bone, including, but not limited to osteopetrosis, osteosclerosis and osteochondrosis.

Thus, in accordance with one aspect of the present invention, there is a method of treating a bone disease comprising: administering to a mammal in need of said treatment a therapeutically effective amount of a compound that lowers NPY level in blood serum, wherein the bone disease is characterized by a decreased bone mass relative to that of corresponding non-diseased bone. Specific embodiments of some of these compounds and methods include, but are not limited to ones that inhibit or lower NPY synthesis or increase NPY breakdown. Among such compounds are antisense, ribozyme or triple helix sequences of a NPY-encoding polypeptide.

In accordance with another aspect of the present invention, there is a method of treating a bone disease comprising: administering to a mammal in need of said treatment a therapeutically effective amount of a compound that lowers NPY level in cerebrospinal fluid, wherein the bone disease is characterized by a decreased bone mass relative to that of corresponding non-diseased bone. Specific embodiments of some of these compounds and methods include, but are not limited to ones that inhibit or lower NPY synthesis or increase NPY breakdown, and compounds that bind NPY in blood.

Particular embodiments of the methods of the invention include, for example, a method of treating a bone disease comprising: administering to a mammal in need of said treatment a therapeutically effective amount of a compound, wherein the bone disease is characterized by a decreased bone mass relative to that of corresponding non-diseased bone, and wherein the compound is selected from the group consisting of compounds which bind NPY in blood, including, but not limited to such compounds as an antibody which specifically binds NPY, and a soluble NPY receptor polypeptide.

In accordance with another aspect of the present invention, there is a method of treating a bone disease comprising: administering to a mammal in need of said treatment a therapeutically effective amount of a compound that lowers the level of extracellular signal-regulated kinase (ERK) activation and inositol phosphate formation, wherein the bone disease is characterized by a decreased bone mass relative to that of corresponding non-diseased bone. Specific embodiments of some of these compounds and methods include, but are not limited to ones that inhibit or lower NPY synthesis or increase NPY breakdown, compounds that bind NPY in blood, and NPY receptor antagonist compounds, such as antibodies which specifically bind NPY, antibodies which specifically bind NPY receptor, compounds that comprise soluble NPY receptor polypeptide sequences and α-alkoxy and α-thioalkoxyamide compositions; dihydropyridine based compounds; substituted benzylamine derivatives; dihydropyrimidone derivatives; naphthimidazolyl derivatives; dimesylate salts); and substituted benzofurans, benzothiophenes or indoles.

In accordance with yet another aspect of the present invention, there is a method of treating a bone disease comprising: administering to a mammal in need of said treatment a therapeutically effective amount of a compound that increases NPY level in blood serum and/or cerebrospinal fluid, wherein the bone disease is characterized by a increased bone mass relative to that of corresponding non-diseased bone. Specific embodiments of some of these compounds and methods include, but are not limited to ones that increase or induce NPY synthesis or decrease NPY breakdown.

In accordance with another aspect of the present invention, there is a method of treating a bone disease comprising: administering to a mammal in need of said treatment a therapeutically effective amount of a compound that increases the level of extracellular signal-regulated kinase (ERK) activation and inositol phosphate formation, wherein the bone disease is characterized by a increased bone mass relative to that of corresponding non-diseased bone. Specific embodiments of some of these compounds and methods include, but are not limited to ones that increase or induce NPY synthesis or decrease NPY breakdown, and NPY receptor agonist compounds such as the NPY agonists or analogs described in U.S. Pat. No. 5,328,899.

In accordance with yet another aspect of the present invention, there is a method of preventing a bone disease comprising: administering to a mammal at risk for the disease a compound that lowers NPY level in blood serum, at a concentration sufficient to prevent the bone disease, wherein the bone disease is characterized by a decreased bone mass relative to that of corresponding non-diseased bone. Specific embodiments of some of these compounds and methods include, but are not limited to ones that inhibit or lower NPY synthesis or increase NPY breakdown. Among such compounds are antisense, ribozyme or triple helix sequences of a NPY-encoding polypeptide.

In accordance with another aspect of the present invention, there is a method of preventing a bone disease comprising: administering to a mammal at risk for the bone disease a compound that lowers NPY level in cerebrospinal fluid, at a concentration sufficient to prevent the bone disease, wherein the bone disease is characterized by a decreased bone mass relative to that of corresponding non-diseased bone. Specific embodiments of some of these compounds and methods include, but are not limited to ones that inhibit or lower NPY synthesis or increase NPY breakdown, and compounds that bind NPY in blood.

Particular embodiments of the methods of the invention include, for example, a method of preventing a bone disease comprising: administering to a mammal at risk for the bone disease a compound at a concentration sufficient to prevent the bone disease, wherein the bone disease is characterized by a decreased bone mass relative to that of corresponding non-diseased bone, and wherein the compound is selected from the group consisting of compounds which bind NPY in blood, including, but not limited to such compounds as an antibody which specifically binds NPY, and a soluble NPY receptor polypeptide.

In accordance with another aspect of the present invention, there is a method of preventing a bone disease comprising: administering to a mammal at risk for the bone disease a compound that lowers the level of extracellular signal-regulated kinase (ERK) activation and inositol phosphate formation, at a concentration sufficient to prevent the bone disease, wherein the bone disease is characterized by a decreased bone mass relative to that of corresponding non-diseased bone. Specific embodiments of some of these compounds and methods include, but are not limited to ones that inhibit or lower NPY synthesis or increase NPY breakdown, compounds that bind NPY in blood, and NPY receptor antagonist compounds, such as antibodies which specifically bind NPY, antibodies which specifically bind NPY receptor, and compounds that comprise soluble NPY receptor polypeptide sequences.

In accordance with yet another aspect of the present invention, there is a method of preventing a bone disease comprising: administering to a mammal at risk for the bone disease a compound that increases NPY level in blood serum and/or cerebrospinal fluid, at a concentration sufficient to prevent the bone disease, wherein the bone disease is characterized by a increased bone mass relative to that of corresponding non-diseased bone. Specific embodiments of some of these compounds and methods include, but are not limited to ones that increase or induce NPY synthesis or decrease NPY breakdown.

In accordance with another aspect of the present invention, there is a method of preventing a bone disease comprising: administering to a mammal at risk for the bone disease a compound that increases the level of extracellular signal-regulated kinase (ERK) activation and inositol phosphate formation, at a concentration sufficient to prevent the bone disease, wherein the bone disease is characterized by a increased bone mass relative to that of corresponding non-diseased bone. Specific embodiments of some of these compounds and methods include, but are not limited to ones that increase or induce NPY synthesis or decrease NPY breakdown, and NPY receptor agonist or analog compounds such as those described in U.S. Pat. No. 5,328,899.

In accordance with another aspect of the present invention, there is a method of preventing a bone disease comprising: administering to a mammal at risk for the disease a compound that increases NPY receptor levels in hypothalamus, at a concentration sufficient to prevent the bone disease, wherein the bone disease is characterized by a increased bone mass relative to that of corresponding non-diseased bone. Specific embodiments of some of these compounds and methods include, but are not limited to ones that increase or induce NPY receptor synthesis or decrease NPY receptor breakdown.

In accordance with yet another aspect of the present invention, there is a method of diagnosing or prognosing a bone disease in a mammal, such as a human, comprising:

(a) measuring NPY levels in blood serum of a mammal, e.g., a mammal suspected of exhibiting or being at risk for the bone disease; and

(b) comparing the level measured in (a) to the NPY level in control blood serum,

so that if the level obtained in (a) is higher than that of the control, the mammal is diagnosed or prognosed as exhibiting or being at risk for the bone disease, wherein the bone disease is characterized by a decreased bone mass relative to that of corresponding non-diseased bone.

In accordance with another aspect of the present invention, there is a method of diagnosing or prognosing a bone disease in a mammal, such as a human, comprising:

(a) measuring NPY levels in cerebrospinal fluid of a mammal, e.g., a mammal suspected of exhibiting or being at risk for the bone disease; and

(b) comparing the level measured in (a) to the NPY level in control cerebrospinal fluid,

so that if the level obtained in (a) is higher than that of the control, the mammal is diagnosed as exhibiting or being at risk for the bone disease, wherein the bone disease is characterized by a decreased bone mass relative to that of corresponding non-diseased bone.

In accordance with yet another aspect of the present invention, there is a method of diagnosing or prognosing a bone disease in a mammal, such as a human, comprising:

(a) measuring NPY levels in blood serum of a mammal, e.g., a mammal suspected of exhibiting or being at risk for the bone disease; and

(b) comparing the level measured in (a) to the NPY level in control blood serum,

so that if the level obtained in (a) is lower than that of the control, the mammal is diagnosed as exhibiting or being at risk for the bone disease, wherein the bone disease is characterized by an increased bone mass relative to that of corresponding non-diseased bone.

In accordance with another aspect of the present invention, there is a method of diagnosing or prognosing a bone disease in a mammal, such as a human, comprising:

(a) measuring NPY levels in cerebrospinal fluid of a mammal, e.g., a mammal suspected of exhibiting or being at risk for the bone disease; and

(b) comparing the level measured in (a) to the NPY level in control cerebrospinal fluid,

so that if the level obtained in (a) is lower than that of the control, the mammal is diagnosed as exhibiting or being at risk for the bone disease, wherein the bone disease is characterized by an increased bone mass relative to that of corresponding non-diseased bone.

In accordance with yet another aspect of the present invention, there is a method of monitoring efficacy of a compound for treating a bone disease in a mammal, such as a human, comprising:

(a) administering the compound to a mammal;

(b) measuring NPY levels in blood serum of the mammal; and

(c) comparing the level measured in (b) to the NPY level in blood serum of the mammal prior to administering the compound,

thereby monitoring the efficacy of the compound, wherein the bone disease is characterized by a decreased bone mass relative to that of corresponding non-diseased bone.

In accordance with another aspect of the present invention, there is a method of monitoring efficacy of a compound for treating a bone disease in a mammal, such as a human, comprising:

(a) administering the compound to a mammal;

(b) measuring NPY levels in cerebrospinal fluid of the mammal; and

(c) comparing the level measured in (b) to the NPY level in cerebrospinal fluid of the mammal prior to administering the compound,

thereby monitoring the efficacy of the compound, wherein the bone disease is characterized by a decreased bone mass relative to that of corresponding non-diseased bone.

In accordance with yet another aspect of the present invention, there is a method of monitoring efficacy of a compound for treating a bone disease in a mammal, such as a human, comprising:

(a) administering the compound to a mammal;

(b) measuring NPY levels in blood serum of the mammal; and

(c) comparing the level measured in (b) to the NPY level in blood serum of the mammal prior to administering the compound,

thereby monitoring the efficacy of the compound, wherein the bone disease is characterized by a increased bone mass relative to that of corresponding non-diseased bone.

In accordance with another aspect of the present invention, there is a method of monitoring efficacy of a compound for treating a bone disease in a mammal, such as a human, comprising:

(a) administering the compound to a mammal;

(b) measuring NPY levels in cerebrospinal fluid of the mammal; and

(c) comparing the level measured in (b) to the NPY level in cerebrospinal fluid of the mammal prior to administering the compound,

thereby monitoring the efficacy of the compound, wherein the bone disease is characterized by a increased bone mass relative to that of corresponding non-diseased bone.

In accordance with another aspect of the present invention, there is a method for identifying a compound to be tested for an ability to modulate (increase or decrease) bone mass in a mammal, comprising:

(a) contacting a test compound with a polypeptide; and

(b) determining whether the test compound binds the polypeptide, so that if the test compound binds the polypeptide, then a compound to be tested for an ability to modulate bone mass is identified, wherein the polypeptide is selected from the group consisting of a neuropeptide Y polypeptide and a neuropeptide Y receptor polypeptide.

In accordance with another aspect of the present invention, there is a method for identifying a compound that modulates (increases or decreases) bone mass in a mammal, comprising:

(a) contacting test compounds with a polypeptide;

(b) identifying a test compound that binds the polypeptide; and

(c) administering the test compound in (b) to a non-human mammal, and determining whether the test compound modulates bone mass in the mammal relative to that of a corresponding bone in an untreated control non-human mammal, wherein the polypeptide is selected from the group consisting of a neuropeptide Y polypeptide and a neuropeptide Y receptor polypeptide, so that if the test compound modulates bone mass, then a compound that modulates bone mass in a mammal is identified.

In accordance with yet another aspect of the present invention, there is a method for identifying a compound to be tested for an ability to modulate (increase or decrease) bone mass in a mammal, comprising:

(a) contacting a test compound with a neuropeptide Y polypeptide and a neuropeptide Y receptor polypeptide for a time sufficient to form neuropeptide Y/neuropeptide Y receptor complexes; and

(b) measuring neuropeptide Y/neuropeptide Y receptor complex level, so that if the level measured differs from that measured in the absence of the test compound, then a compound to be tested for an ability to modulate bone mass is identified.

In accordance with another aspect of the present invention, there is a method for identifying a compound to be tested for an ability to decrease bone mass in a mammal, comprising:

(a) contacting a test compound with a cell which expresses a functional neuropeptide Y receptor; and

(b) determining whether the test compound activates the neuropeptide Y receptor, wherein if the compound activates the neuropeptide Y receptor a compound to be tested for an ability to decrease bone mass in a mammal is identified.

In accordance with another aspect of the present invention, there is a method for identifying a compound that decreases bone mass in a mammal, comprising:

(a) contacting a test compound with a cell that expresses a functional neuropeptide Y receptor, and determining whether the test compound activates the neuropeptide Y receptor;

(b) administering a test compound identified in (a) as activating the neuropeptide Y receptor to a non-human animal, and determining whether the test compound decreases bone mass of the animal relative to that of a corresponding bone of a control non-human animal, so that if the test compound decreases bone mass, then a compound that decreases bone mass in a mammal is identified.

In accordance with another aspect of the present invention, there is a method for identifying a compound to be tested for an ability to increase bone mass in a mammal, comprising:

(a) contacting a neuropeptide Y polypeptide and a test compound with a cell that expresses a functional neuropeptide Y receptor; and

(b) determining whether the test compound lowers activation of the neuropeptide Y receptor relative to that observed in the absence of the test compound; wherein a test compounds that lowers activation of the neuropeptide Y receptor is identified as a compound to be tested for an ability to increase bone mass in a mammal.

In accordance with yet another aspect of the present invention, there is a method for identifying a compound that increases bone mass in a mammal, comprising:

(a) contacting a neuropeptide Y polypeptide and a test compound with a cell that expresses a functional neuropeptide Y receptor, and determining whether the test compound decreases activation of the neuropeptide Y receptor;

(b) administering a test compound identified in (a) as decreasing neuropeptide Y receptor to a non-human animal, and determining whether the test compound increases bone mass of the animal relative to that of a corresponding bone of a control non-human animal, so that if the test compound increases bone mass, then a compound that increases bone mass in a mammal is identified.

The present invention also provides pharmaceutical compositions which can be used to treat and/or prevent bone diseases.

Other and further objects, features and advantages would be apparent and eventually more readily understood by reading the following specification and by reference to the accompanying drawings forming a part thereof, or any examples of the presently preferred embodiments of the invention are given for the purpose of the disclosure.

3.1 Definitions

The following terms used herein shall have the meaning indicated:

NPY, as used herein, is defined as neuropeptide Y, preferably human neuropeptide Y. Neuropeptide Y (NPY) is a member of the pancreatic polypeptide family. It is to be understood that the term NPY, as used herein is intended to encompass not only neuropeptide Y but also its peptide relatives in the pancreatic polypeptide family, e.g., peptide YY (PYY), and pancreatic polypeptide (PP).

Neuropeptide Y receptor (“NPY receptor” or “NPY-R”), as used herein, is defined as a receptor, preferably a human receptor, that binds endogenous NPY under physiological conditions. NPY receptors are G protein-coupled receptors including, but not limited to subtypes known as Y1, Y2, Y3, Y4, or Y5 (or PP).

Bone disease, as used herein, refers to any bone disease or state which results in or is characterized by loss of health or integrity to bone and includes, but is not limited to, osteoporosis, osteopenia, faulty bone formation or resorption, Paget's disease, fractures and broken bones, bone metastasis, osteopetrosis, osteosclerosis and osteochondrosis. More particularly, bone diseases which can be treated and/or prevented in accordance with the present invention include bone diseases characterized by a decreased bone mass relative to that of corresponding non-diseased bone (e.g., osteoporosis, osteopenia and Paget's disease), and bone diseases characterized by an increased bone mass relative to that of corresponding non-diseased bone (e.g., osteopetrosis, osteosclerosis and osteochondrosis). Prevention of bone disease includes actively intervening as described herein prior to onset to prevent the disease. Treatment of bone disease encompasses actively intervening after onset to slow down, ameliorate symptoms of, or reverse the disease or situation. More specifically, treating, as used herein, refers to a method that modulates bone mass to more closely resemble that of corresponding non-diseased bone (that is a corresponding bone of the same type, e.g., long, vertebral, etc.) in a non-diseased state.

NPY receptor antagonist, as used herein, refers to a factor which neutralizes or impedes or otherwise reduces the action or effect of a NPY receptor. Such antagonists can include compounds that bind NPY or that bind NPY receptor. Such antagonists can also include compounds that neutralize, impede or otherwise reduce NPY receptor output, that is, intracellular steps in the NPY signaling pathway following binding of NPY to the NPY receptor, i.e., downstream events that affect NPY/NPY receptor signaling, that do not occur at the receptor/ligand interaction level. NPY receptor antagonists may include, but are not limited to proteins, antibodies, small organic molecules or carbohydrates, such as, for example, antibodies which specifically bind NPY, antibodies which specifically bind NPY receptor, and compounds that comprise soluble NPY receptor polypeptide sequences. Additional NPY receptor antagonists include, but are not limited to α-alkoxy and α-thioalkoxyamide compositions; dihydropyridine based compounds; substituted benzylamine derivatives; dihydropyrimidone derivatives; naphthimidazolyl derivatives; dimesylate salts); and substituted benzofurans, benzothiophenes or indoles.

NPY receptor agonist, as used herein, refers to a factor which activates, induces or otherwise increases the action or effect of a NPY receptor. Such agonists can include compounds that bind NPY or that bind NPY receptor. Such antagonists can also include compounds that activate, induce or otherwise increase NPY receptor output, that is, intracellular steps in the NPY signaling pathway following binding of NPY to the NPY receptor, i.e., downstream events that affect NPY/NPY receptor signaling, that do not occur at the receptor/ligand interaction level. NPY receptor agonists may include, but are not limited to proteins, antibodies, small organic molecules or carbohydrates, such as, for example, NPY, NPY analogs, antibodies which specifically bind and activate NPY and NPY receptor agonist and analog compounds such as those described in U.S. Pat. No. 5,328,899.

An agent is said to be administered in a “therapeutically effective amount” if the amount administered results in a desired change in the physiology of a recipient mammal, e.g., results in an increase or decrease in bone mass relative to that of a corresponding bone in the diseased state; that is, results in treatment, i.e., modulates bone mass to more closely resemble that of corresponding non-diseased bone (that is a corresponding bone of the same type, e.g., long, vertebral, etc.) in a non-diseased state.

ECD, as used herein, refers to extracellular domain.

TM, as used herein, refers to transmembrane domain.

CD, as used herein, refers to cytoplasmic domain.

### 4 BRIEF DESCRIPTION OF THE FIGURES

FIG. 1. NPY icv Infusions Affect Bone Mass. Histological comparison of vertebrae of 4 month-old wt mice infused centrally with PBS or NPY. NPY icv infusion causes a decrease in bone mass and bone volume. Underlined numbers indicate a statistically significant difference between experimental and control groups of mice (p<0.05).

The figure is not necessarily to scale and certain features mentioned may be exaggerated in scale or shown in schematic form in the interest of clarity and conciseness.

### 5 DETAILED DESCRIPTION OF THE INVENTION

Various aspects of the present invention are presented in detail herein.

5.1 NPY and NPY Receptor Proteins, Polypeptides, Nucleic Acids and Antibodies

Neuropeptide Y (“NPY”) and neuropeptide Y receptor (“NPY receptor”) proteins and nucleic acids (sense and antisense) can be utilized as part of the therapeutic, diagnostic, prognostic and screening methods of the present invention. For example, NPY and/or NPY receptor proteins, polypeptides and peptide fragments, mutated, truncated or deleted forms of NPY or NPY receptor, including, but not limited to, soluble derivatives such as peptides or polypeptides corresponding to one or more NPY receptor ECDs; truncated NPY receptor polypeptides lacking one or more ECD or TM; and NPY and NPY receptor fusion protein products (such as NPY receptor-Ig fusion proteins, that is, fusions of the NPY receptor or a domain of the NPY receptor, to an IgFc domain) can be utilized.

Sequences of NPY and NPY receptors, including human NPY and NPY receptors, are well known. Neuropeptide Y (NPY) is a 36-amino acid peptide neurotransmitter that is located throughout the central and peripheral nervous systems. Tatemoto, Proc. Natl. Acad. Sci. USA 79, 5485 (1982); Hazlewood, Proc. Soc. Exp. Biol. Med. 202, 44 (1993). Sequences of NPY are well known. See e.g., Takeuchi,T., et al., 1986, J. Clin. Invest., 77 (3):1038-1041, Colmers and Wahlestedt, The Biology of Neuropeptide Y and Related Peptides (Humana Press, Totowa, N.J., 1993), and Hazlewood, Proc. Soc. Exp. Biol. Med. 202, 44 (1993). NPY receptors are protein-coupled receptors including, but not limited to subtypes known as Y1, Y2, Y3, Y4, or Y5 (or PP), sequences of which are well known to those of skill in the art. See, e.g., WO 93/09227, WO 93/24515, Larhammar et al., J. Biol. Chem. 267:10935 (1992); Eva et al., 1990, FEBS Lett. 271:81; and Eva et al., 1990, FEBS Lett. 314:286 (NPY1); WO 95/21245; Rose et al., J. Biol. Chem. 270:22661 (1995) (NPY2); U.S. Pat. No. 5,965,392 (NPY3); WO 95/17906 (NPY4); and U.S. Pat. No. 5,968,819 (NPY5).

For example, peptides and polypeptides corresponding to NPY or to one or more domains of the NPY receptor (e.g., ECD, TM or CD), truncated or deleted NPY or NPY receptors (e.g., NPY receptor in which the TM and/or CD is deleted) as well as fusion proteins in which the full length NPY or NPY receptor, an NPY or NPY receptor peptide or truncated NPY or NPY receptor (e.g., an NPY receptor ECD, TM or CD domain) is fused to a heterologous, unrelated protein are also within the scope of the invention and can be utilized and designed on the basis of such NPY and NPY receptor nucleotide and NPY and NPY receptor amino acid sequences which are known to those of skill in the art. Preferably, NPY polypeptides can bind NPY receptor under standard physiological and/or cell culture conditions. Likewise, preferably leptor receptor polypeptides can bind NPY under standard physiological and/or cell culture conditions. Thus, at a minimum, NPY receptor polypeptides comprise a NPY amino acid sequence sufficient for NPY receptor binding, that is for NPY/NPY receptor complex formation and likewise, at a minimum, NPY receptor polypeptides comprise a NPY receptor ECD sequence sufficient for NPY binding.

With respect to NPY receptor peptides, polypeptides, fusion peptides and fusion polypeptides comprising all or part of an NPY receptor ECD, such peptides include soluble NPY receptor polypeptides. Preferably, such soluble NPY receptor polypeptides can bind NPY under standard physiological and/or cell culture conditions. Thus, at a minimum, such soluble NPY receptor polypeptides comprise an NPY receptor ECD sequence sufficient for NPY binding.

Fusion proteins include, but are not limited to, IgFc fusions which stabilize the soluble NPY receptor protein or peptide and prolong half-life in vivo; or fusions to any amino acid sequence that allows the fusion protein to be anchored to the cell membrane, allowing the ECD to be exhibited on the cell surface; or fusions to an enzyme, fluorescent protein, or luminescent protein which provide a marker or reporter function, useful e.g, in screening and/or diagnostic methods of the invention.

While the NPY and NPY-R polypeptides and peptides can be chemically synthesized (e.g., see Creighton, 1983, Proteins: Structures and Molecular Principles, W. H. Freeman & Co., N.Y.), large polypeptides derived from NPY and NPY-R and full length NPY and NPY-R may advantageously be produced by recombinant DNA technology using techniques well known in the art for expressing nucleic acid containing NPY and NPY-R gene sequences and/or coding sequences. Such methods also can be used to construct expression vectors containing the NPY and NPY-R nucleotide sequences. These methods include, for example, in vitro recombinant DNA techniques, synthetic techniques, and in vivo genetic recombination. See, Sambrook et al., 1989, Molecular Cloning, A Laboratory Manual, Second Edition, Cold Spring Harbor Press, N.Y., and Ausabel et al., 1989, Current Protocols in Molecular Biology, Green Publishing Associates and Wiley Interscience, N.Y., each of which is incorporated herein by reference in its entirety. Alternatively, RNA capable of encoding NPY and NPY-R nucleotide sequences may be chemically synthesized using, for example, synthesizers. See, for example, the techniques described in “Oligonucleotide Synthesis”, 1984, Gait, M. J. ed., IRL Press, Oxford, which is incorporated by reference herein in its entirety.

A variety of host-expression vector systems may be utilized to express the NPY and NPY-R nucleotide sequences of the invention. Where the NPY and NPY-R peptide or polypeptide is a soluble derivative (e.g., NPY-R peptides corresponding to the ECD; truncated or deleted NPY-R in which the TM and/or CD are deleted) the peptide or polypeptide can be recovered from the culture, ie., from the host cell in cases where the NPY-R peptide or polypeptide is not secreted, and from the culture media in cases where the NPY-R peptide or polypeptide is secreted by the cells. However, the expression systems also encompass engineered host cells that express NPY and NPY-R or functional equivalents in situ, i.e., anchored in the cell membrane. Purification or enrichment of NPY or NPY-R from such expression systems can be accomplished using appropriate detergents and lipid micelles and methods well known to those skilled in the art. However, such engineered host cells themselves may be used in situations where it is important not only to retain the structural and functional characteristics of NPY and NPY-R, but to assess biological activity, e.g., in drug screening assays.

The expression systems that may be used for purposes of the invention include, but are not limited to, microorganisms such as bacteria (e.g., E. coli, B. subtilis) transformed with recombinant bacteriophage DNA, plasmid DNA or cosmid DNA expression vectors containing NPY or NPY-R nucleotide sequences; yeast (e.g., Saccharomyces, Pichia) transformed with recombinant yeast expression vectors containing the nucleotide sequences; insect cell systems infected with recombinant virus expression vectors (e.g., baculovirus) containing the sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the nucleotide sequences; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the NPY or NPY-R gene product being expressed. For example, when a large quantity of such a protein is to be produced, for the generation of pharmaceutical compositions of NPY or NPY-R protein or for raising antibodies to NPY or NPY-R protein, for example, vectors which direct the expression of high levels of fusion protein products that are readily purified may be desirable. Such vectors include, but are not limited to, the E. coli expression vector pUR278 (Ruther et al., 1983, EMBO J. 2:1791), in which the NPY or NPY-R coding sequence may be ligated individually into the vector in frame with the lacZ coding region so that a fusion protein is produced; pIN vectors (Inouye & Inouye, 1985, Nucleic Acids Res. 13:3101-3109; Van Heeke & Schuster, 1989, J. Biol. Chem. 264:5503-5509); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The PGEX vectors are designed to include thrombin or factor Xa protease cleavage sites so that the cloned target gene product can be released from the GST moiety.

In an insect system, Autographa californica nuclear polyhidrosis virus (AcNPV) is used as a vector to express foreign genes. The virus grows in Spodoptera frugiperda cells. The NPY or NPY-R gene coding sequence may be cloned individually into non-essential regions (for example the polyhedrin gene) of the virus and placed under control of an AcNPV promoter (for example the polyhedrin promoter). Successful insertion of an NPY or NPY-R gene coding sequence will result in inactivation of the polyhedrin gene and production of non-occluded recombinant virus, (i.e., virus lacking the proteinaceous coat coded for by the polyhedrin gene). These recombinant viruses are then used to infect Spodoptera frugiperda cells in which the inserted gene is expressed. (E.g., see Smith et al., 1983, J. Virol. 46: 584; Smith, U.S. Pat. No. 4,215,051).

In mammalian host cells, a number of viral-based expression systems may be utilized. In cases where an adenovirus is used as an expression vector, the NPY or NPY-R nucleotide sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the NPY or NPY-R gene product in infected hosts. (E.g., See Logan & Shenk, 1984, Proc. Natl. Acad. Sci. USA 81:3655-3659). Specific initiation signals may also be required for efficient translation of inserted NPY or NPY-R nucleotide sequences. These signals include the ATG initiation codon and adjacent sequences. In cases where entire NPY or NPY-R genes or cDNAs, including their own initiation codons and adjacent sequences, are inserted into the appropriate expression vector, no additional translational control signals may be needed. However, in cases where only a portion of the coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (See Bittner et al., 1987, Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, WI38, and in particular, cell lines of the central and peripheral nervous systems.

For long-term, high-yield production of recombinant proteins, stable expression is preferred. For example, cell lines which stably express the NPY or NPY-R sequences may be engineered. Rather than using expression vectors which contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the NPY or NPY-R gene products. Such engineered cell lines may be particularly useful in screening and evaluation of compounds that affect the endogenous activity of NPY and NPY-R gene products.

A number of selection systems may be used, including but not limited to, the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in tk⁻, hgprt⁻ or aprt⁻ cells, respectively. Also, antimetabolite resistance can be used as the basis of selection for the following genes: dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147).

Alternatively, any fusion protein may be readily purified by utilizing an antibody specific for the fusion protein being expressed. For example, a system described by Janknecht et al. allows for the ready purification of non-denatured fusion proteins expressed in human cell lines (Janknecht, et al., 1991, Proc. Natl. Acad. Sci. USA 88: 8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

The NPY and NPY-R gene products can also be expressed in transgenic animals. Animals of any species, including, but not limited to, mice, rats, rabbits, guinea pigs, pigs, micro-pigs, goats, and non-human primates, e.g., baboons, monkeys, and chimpanzees may be used to generate the transgenic animals.

Any technique known in the art may be used to introduce the NPY or NPY receptor transgene into animals or to “knock-out” or inactivate endogenous NPY or NPY receptor to produce the founder lines of transgenic animals. Such animals can be utilized as part of the screening methods of the invention, and cells and/or tissues from such animals can be obtained for generation of additional compositions (e.g., cell lines, tissue culture systems) that can also be utilized as part of the screening methods of the invention.

Techniques for generation of such animals are well known to those of skill in the art and include, but are not limited to, pronuclear microinjection (Hoppe, P. C. and Wagner, T. E., 1989, U.S. Pat. No. 4,873,191); retrovirus mediated gene transfer into germ lines (Van der Putten et al., 1985, Proc. Natl. Acad. Sci., USA 82:6148-6152); gene targeting in embryonic stem cells (Thompson et al., 1989, Cell 56:313-321); electroporation of embryos (Lo, 1983, Mol Cell. Biol. 3:1803-1814); and sperm-mediated gene transfer (Lavitrano et al., 1989, Cell 57:717-723); etc. For a review of such techniques, see Gordon, 1989, Transgenic Animals, Intl. Rev. Cytol. 115:171-229, which is incorporated by reference herein in its entirety.

With respect to transgenic animals containing a transgenic NPY and/or NPY receptor, such animals can carry an NPY or NPY receptor transgene in all their cells. Alternatively, such animals can carry the transgene or transgenes in some, but not all their cells, i.e., mosaic animals. The transgene may be integrated as a single transgene or in concatamers, e.g., head-to-head tandems or head-to-tail tandems. The transgene may also be selectively introduced into and activated in a particular cell type by following, for example, the teaching of Lasko et al. (Lasko, M. et al., 1992, Proc. Natl.Acad. Sci. USA 89: 6232-6236). The regulatory sequences required for such a cell-type specific activation will depend upon the particular cell type of interest, and will be apparent to those of skill in the art. When it is desired that the transgene be integrated into the chromosomal site of the endogenous gene, gene targeting is preferred. Briefly, when such a technique is to be utilized, vectors containing some nucleotide sequences homologous to the endogenous NPY or NPY-R gene are designed for the purpose of integrating, via homologous recombination with chromosomal sequences, into and disrupting the function of the nucleotide sequence of the endogenous NPY or NPY-R gene, respectively. The transgene may also be selectively introduced into a particular cell type, thus inactivating the endogenous NPY or NPY-R gene in only that cell type, by following, for example, the teaching of Gu et al. (Gu, et al., 1994, Science 265: 103-106). The regulatory sequences required for such a cell-type specific inactivation will depend upon the particular cell type of interest, and will be apparent to those of skill in the art.

Once transgenic animals have been generated, the expression of the recombinant gene may be assayed utilizing standard techniques. Initial screening may be accomplished by Southern blot analysis or PCR techniques to analyze animal tissues to assay whether integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and RT-PCR. Samples of NPY and NPY-R gene-expressing tissue, may also be evaluated immunocytochemically using antibodies specific for the transgene product.

5.1.1 Antibodies to NPY and NPY-R Proteins

Antibodies that specifically recognize and bind to one or more epitopes of NPY or NPY receptor, or epitopes of conserved variants of NPY or NPY receptor, or peptide fragments of NPY or NPY receptor can be utilized as part of the methods of the present invention. Such antibodies include, but are not limited to, polyclonal antibodies, monoclonal antibodies (mAbs), human, humanized or chimeric antibodies, single chain antibodies, Fab fragments, F(ab′)₂ fragments, fragments produced by a Fab expression library, anti-idiotypic (anti-Id) antibodies and epitope-binding fragments of any of the above.

Such antibodies may be used, for example, as part of the diagnostic or prognostic methods of the invention for diagnosing a bone disease in a mammal by measuring NPY levels in the mammal, e.g., NPY levels in blood serum or cerebrospinal fluid of the mammal. Such antibodies may also be utilized in conjunction with, for example, compound screening schemes, as described below, for the evaluation of the effect of test compounds on expression and/or activity of the NPY or NPY receptor gene product. Additionally, such antibodies can be used in therapeutic and preventative methods of the invention. For example, such antibodies can correspond to NPY receptor agonists or antagonists. Further, such antibodies can be administered to lower NPY levels in the brain, as assayed by NPY levels in cerebrospinal fluid. In addition, such antibodies can be utilized to lower NPY levels by increasing the rate at which NPY is removed from circulation (e.g., can speed NPY breakdown), or can be used to lower NPY receptor levels, including lowering cells expressing NPY receptor, by increasing the rate at which NPY receptor (and cells expressing NPY receptor) breaks down or is degraded.

For the production of antibodies, various host animals may be immunized by injection with NPY or NPY receptor, an NPY or NPY receptor peptide (e.g., for NPY receptor, one corresponding with a functional domain of the receptor, such as ECD, TM or CD), truncated NPY or NPY receptor polypeptides (e.g., for NPY receptor, in which one or more domains, e.g., the TM or CD, has been deleted), functional equivalents of NPY or NPY receptor or mutants of NPY or NPY receptor. Such host animals may include, but are not limited to, rabbits, mice, and rats, to name but a few. Various adjuvants may be used to increase the immunological response, depending on the host species, including but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Polyclonal antibodies are heterogeneous populations of antibody molecules derived from the sera of the immunized animals.

Monoclonal antibodies, which are homogeneous populations of antibodies to a particular antigen, may be obtained by any technique which provides for the production of antibody molecules by continuous cell lines in culture. These include, but are not limited to, the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al, 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Fremont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al. (1994) Bio/technology 12:899-903).

Alternatively, techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can be adapted to produce single chain antibodies against NPY and NPY receptor gene products. Single chain antibodies are formed by linking the heavy and light chain fragments of the Fv region via an amino acid bridge, resulting in a single chain polypeptide.

Antibody fragments which recognize specific epitopes may be generated by known techniques. For example, such fragments include, but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science, 246:1275-1281) to allow rapid and easy identification of monoclonal Fab fragments with the desired specificity.

Antibodies to NPY or NPY receptor can, in turn, be utilized to generate anti-idiotype antibodies that “mimic” NPY or NPY receptor, using techniques well known to those skilled in the art. (See, e.g., Greenspan & Bona, 1993, FASEB J 7(5):437-444; and Nissinoff, 1991, J. Immunol. 147(8):2429-2438). For example, antibodies which bind to the NPY receptor ECD and competitively inhibit the binding of NPY to the NPY receptor can be used to generate anti-idiotypes that “mimic” the ECD and, therefore, bind and neutralize NPY. Such neutralizing anti-idiotypes or Fab fragments of such anti-idiotypes can be used in therapeutic regimens to neutralize NPY and treat bone disease characterized by a decreased bone mass relative to a corresponding non-diseased bone.

5.2 Diagnosis and Prognosis of Bone Disease and Compound/Patient Monitoring

A variety of methods can be employed for the diagnostic and prognostic evaluation of bone diseases or states, including, but not limited to, osteoporosis, osteopenia, faulty bone formation or resorption, Paget's disease, fractures and broken bones, bone metastasis, osteopetrosis, osteosclerosis and osteochondrosis and for the identification of subjects having a predisposition to such diseases or states.

In particular, bone diseases which can be diagnosed or prognosed in accordance with the present invention include bone diseases characterized by a decreased bone mass relative to that of corresponding non-diseased bone, including, but not limited to osteoporosis, osteopenia and Paget's disease.

Thus, in accordance with this aspect of the present invention, there is a method of diagnosing or prognosing a bone disease in a mammal, such as a human, comprising:

(a) measuring neuropeptide Y levels in blood serum of a mammal, e.g., a mammal suspected of exhibiting or being at risk for the bone disease; and

(b) comparing the level measured in (a) to the neuropeptide Y level in control blood serum,

so that if the level NPY obtained in (a) is higher than that of the control, the mammal is diagnosed as exhibiting or being at risk for the bone disease, wherein the bone disease is characterized by a decreased bone mass relative to that of corresponding non-diseased bone.

Alternatively, there is a method of diagnosing or prognosing a bone disease in a mammal, such as a human, comprising:

(a) measuring neuropeptide Y levels in cerebrospinal fluid of a mammal, e.g., a mammal suspected of exhibiting or being at risk for the bone disease; and

(b) comparing the level measured in (a) to the neuropeptide Y level in control cerebrospinal fluid,

so that if the level NPY obtained in (a) is higher than that of the control, the mammal is diagnosed as exhibiting or being at risk for the bone disease, wherein the bone disease is characterized by a decreased bone mass relative to that of corresponding non-diseased bone.

Further, bone diseases which can be diagnosed or prognosed in accordance with the present invention also include bone diseases characterized by an increased bone mass relative to that of corresponding non-diseased bone, including, but not limited to osteopetrosis, osteosclerosis and osteochondrosis.

Thus, in accordance with this aspect of the present invention, there is a method of diagnosing or prognosing a bone disease in a mammal, such as a human, comprising:

(a) measuring neuropeptide Y levels in blood serum of a mammal, e.g., a mammal suspected of exhibiting or being at risk for the bone disease; and

(b) comparing the level measured in (a) to the neuropeptide Y level in control blood serum,

so that if the level NPY obtained in (a) is lower than that of the control, the mammal is diagnosed as exhibiting or being at risk for the bone disease, wherein the bone disease is characterized by an increased bone mass relative to that of corresponding non-diseased bone.

Alternatively, there is a method of diagnosing or prognosing a bone disease in a mammal, such as a human, comprising:

(a) measuring neuropeptide Y levels in cerebrospinal fluid of a mammal, e.g., a mammal suspected of exhibiting or being at risk for the bone disease; and

(b) comparing the level measured in (a) to the neuropeptide Y level in control cerebrospinal fluid,

so that if the level NPY obtained in (a) is lower than that of the control, the mammal is diagnosed as exhibiting or being at risk for the bone disease, wherein the bone disease is characterized by an increased bone mass relative to that of corresponding non-diseased bone.

Additionally, methods are provided for the diagnostic monitoring of patients undergoing clinical evaluation for the treatment of bone disease, and for monitoring the efficacy of compounds in clinical trials.

Thus, yet another aspect of the present, invention, there is a method of monitoring efficacy of a compound for treating a bone disease in a mammal, such as a human, comprising:

(a) administering the compound to a mammal;

(b) measuring neuropeptide Y levels in blood serum of the mammal; and

(c) comparing the level measured in (b) to the neuropeptide Y level in blood serum of the mammal prior to administering the compound,

thereby monitoring the efficacy of the compound, wherein the bone disease is characterized by a decreased bone mass relative to that of corresponding non-diseased bone. Preferred compounds are ones that increase neuropeptide Y levels relative to that NPY observed prior to administration.

In accordance with another aspect of the present invention, there is a method of monitoring efficacy of a compound for treating a bone disease in a mammal, such as a human, comprising:

(a) administering the compound to a mammal;

(b) measuring neuropeptide Y levels in cerebrospinal fluid of the mammal; and

(c) comparing the level measured in (b) to the neuropeptide Y level in cerebrospinal fluid of the mammal prior to administering the compound,

thereby monitoring the efficacy of the compound, wherein the bone disease is characterized by a decreased bone mass relative to that of corresponding non-diseased bone. Preferred compounds are ones that increase neuropeptide Y levels relative to that NPY observed prior to administration.

In accordance with yet another aspect of the present invention, there is a method of monitoring efficacy of a compound for treating a bone disease in a mammal, such as a human, comprising:

(a) administering the compound to a mammal;

(b) measuring neuropeptide Y levels in blood serum of the mammal; and

(c) comparing the level measured in (b) to the neuropeptide Y level in blood serum of the mammal prior to administering the compound,

thereby monitoring the efficacy of the compound, wherein the bone disease is characterized by a increased bone mass relative to that of corresponding non-diseased bone. Preferred compounds are ones that decrease neuropeptide Y levels relative to that NPY observed prior to administration.

In accordance with another aspect of the present invention, there is a method of monitoring efficacy of a compound for treating a bone disease in a mammal, such as a human, comprising:

(a) administering the compound to a mammal;

(b) measuring neuropeptide Y levels in cerebrospinal fluid of the mammal; and

(c) comparing the level measured in (b) to the neuropeptide Y level in cerebrospinal fluid of the mammal prior to administering the compound,

thereby monitoring the efficacy of the compound, wherein the bone disease is characterized by a increased bone mass relative to that of corresponding non-diseased bone. Preferred compounds are ones that decrease neuropeptide Y levels relative to that NPY observed prior to administration.

Methods such as these can also be utilized for monitoring of patients undergoing lcinical evaluation for treatment of bone disease. Generally, such methods further include a monitoring of bone mass relative to a corresponding non-diseased bone.

Methods described herein may, for example, utilize reagents such as the NPY and NPY receptor nucleotide sequences described above and known to those of skill in the art, and NPY and NPY receptor antibodies, as described, in Section 5.1.1. NPY is typically located throughout the central and peripheral nervous systems. As such, such reagents may be used, for example, for: (1) the detection of the presence of NPY and NPY receptor gene mutations, or the detection of either over- or under-expression of NPY or NPY receptor mRNA relative to the non-bone diseased states, e.g., in a mammal's blood serum or in cerebrospinal fluid; (2) the detection of either an over- or an under-abundance of NPY or NPY receptor gene product relative to the non-bone diseased states, e.g., in a mammal's blood serum or in cerebrospinal fluid; and (3) the detection of perturbations or abnormalities in the signal transduction pathway mediated by NPY or NPY receptor. Alternatively, levels of extracellular signal-regulated kinase (ERK) activation and inositol phosphate formation can be measured relative to levels NPY observed in a corresponding control sample or mammal. ERK activation and inositol phosphate formation are biochemical events which occurs following binding of NPY to NPY receptor.

The methods described herein may be performed in conjunction with, prior to, or subsequent to techniques for measuring bone mass. For example, upon identifying a mammal (e.g., human) exhibiting higher or lower levels of neuropeptide Y (e.g., in blood serum or cerebospinal fluid) relative to that of a corresponding control sample, bone mass of the individual can be measured to further clarify whether the mammal exhibits increased or decreased bone mass relative to a corresponding non-diseased bone. If no abnormal bone mass is NPY observed, the mammal can be considered to be at risk for developing disease, while is an abnormal bone mass is observed, the mammal exhibits the bone disease.

Among the techniques well known to those of skill in the art for measuring bone mass are ones that include, but are not limited to, skeletal X-ray, which shows the lucent level of bone (the lower the lucent level, the higher the bone mass); classical bone histology (e.g., bone vlume, number and aspects of trabeculi/trabeculations, numbers of osteoblast relative to controls and/or relative to osteoclasts); and dual energy X-ray absorptometry (DEXA) (Levis and Altman, 1998, Arthritis and Rheumatism, 41:577-587) which measures bone mass and is commonly used in osteoporosis.

The methods described herein may further be used to diagnose individuals at risk for bone disease. Such individuals include, but are not limited to, peri-menopausal women (as used herein, this tem is meant to encompass a time frame from approximately 6 months prior to the onset of menopause to approximately 18 months subsequent to menopause) and patients undergoing treatment with corticosteroids, especially long-term corticosteroid treatment.

The methods described herein may be performed, for example, by utilizing pre-packaged diagnostic kits comprising at least one specific NPY or NPY receptor nucleotide sequence or NPY or NPY receptor antibody reagent, which may be conveniently used, e.g., in clinical settings, to diagnose patients exhibiting bone diseases.

For the detection of NPY or NPY receptor mutations, any nucleated cell can be used as a starting source for genomic nucleic acid. For the detection of NPY or NPY receptor gene expression or gene products, any cell type or tissue in which the NPY or NPY receptor gene is expressed.

Nucleic acid-based detection techniques are described below, in Section 5.2.1. Peptide detection techniques are described below, in Section 5.2.2.

5.2.1 Detection of NPY and NPY receptor Gene and Transcripts

Mutations within the NPY and NPY receptor gene can be detected by utilizing a number of techniques. Nucleic acid from any nucleated cell can be used as the starting point for such assay techniques, and may be isolated according to standard nucleic acid preparation procedures which are well known to those of skill in the art.

DNA may be used in hybridization or amplification assays of biological samples to detect abnormalities involving NPY or NPY receptor gene structure, including point mutations, insertions, deletions and chromosomal rearrangements. Such assays may include, but are not limited to, Southern analyses, single stranded conformational polymorphism analyses (SSCP), and PCR analyses.

Such diagnostic methods for the detection of NPY or NPY receptor gene-specific mutations can involve for example, contacting and incubating nucleic acids including recombinant DNA molecules, cloned genes or degenerate variants thereof, NPY obtained from a sample, e.g., derived from a patient sample or other appropriate cellular source, with one or more labeled nucleic acid reagents including recombinant DNA molecules, cloned genes or degenerate variants thereof, under conditions favorable for the specific annealing of these reagents to their complementary sequences within the NPY or NPY receptor gene, respectively. Preferably, the lengths of these nucleic acid reagents are at least 15 to 30 nucleotides. After incubation, all non-annealed nucleic acids are removed from the nucleic acid:NPY/NPY receptor molecule hybrid. The presence of nucleic acids which have hybridized, if any such molecules exist, is then detected. Using such a detection scheme, the nucleic acid from the cell type or tissue of interest can be immobilized, for example, to a solid support such as a membrane, or a plastic surface such as that on a microtiter plate or polystyrene beads. In this case, after incubation, non-annealed, labeled nucleic acid reagents are easily removed. Detection of the remaining, annealed, labeled NPY or NPY receptor nucleic acid reagents is accomplished using standard techniques well-known to those in the art. The NPY or NPY receptor gene sequences to which the nucleic acid reagents have annealed can be compared to the annealing pattern expected from a normal NPY or NPY receptor gene sequence in order to determine whether an NPY or NPY receptor gene mutation is present.

Alternative diagnostic methods for the detection of NPY or NPY receptor gene specific nucleic acid molecules, in patient samples or other appropriate cell sources, may involve their amplification, e.g., by PCR (the experimental embodiment set forth in Mullis, K. B., 1987, U.S. Pat. No. 4,683,202), followed by the detection of the amplified molecules using techniques well known to those of skill in the art. The resulting amplified sequences can be compared to those which would be expected if the nucleic acid being amplified contained only normal copies of the NPY or NPY receptor gene in order to determine whether an NPY or NPY receptor gene mutation exists.

Additionally, well-known genotyping techniques can be performed to identify individuals carrying NPY or NPY receptor gene mutations. Such techniques include, for example, the use of restriction fragment length polymorphisms (RFLPs), which involve sequence variations in one of the recognition sites for the specific restriction enzyme used.

Additionally, improved methods for analyzing DNA polymorphisms which can be utilized for the identification of NPY or NPY receptor gene mutations have been described which capitalize on the presence of variable numbers of short, tandemly repeated DNA sequences between the restriction enzyme sites. For example, Weber (U.S. Pat. No. 5,075,217, which is incorporated herein by reference in its entirety) describes a DNA marker based on length polymorphisms in blocks of (dC-dA)n-(dG-dT)n short tandem repeats. The average separation of (dC-dA)n-(dG-dT)n blocks. is estimated to be 30,000-60,000 bp. Markers which are so closely spaced exhibit a high frequency co-inheritance, and are extremely useful in the identification of genetic mutations, such as, for example, mutations within the NPY or NPY receptor gene, and the diagnosis of diseases and disorders related to NPY or NPY receptor mutations.

Also, Caskey et al. (U.S. Pat. No. 5,364,759, which is incorporated herein by reference in its entirety) describe a DNA profiling assay for detecting short tri and tetra nucleotide repeat sequences. The process includes extracting the DNA of interest, such as the NPY or NPY receptor gene, amplifying the extracted DNA, and labeling the repeat sequences to form a genotypic map of the individual's DNA.

The level of NPY or NPY receptor gene expression can also be assayed by detecting and measuring NPY or NPY receptor transcription, respectively. For example, RNA from a cell type or tissue known, or suspected to express the NPY or NPY receptor gene, such as brain, may be isolated and tested utilizing hybridization or PCR techniques such as are described, above. The isolated cells can be derived from cell culture or from a patient. The analysis of cells taken from culture may be a necessary step in the assessment of cells to be used as part of a cell-based gene therapy technique or, alternatively, to test the effect of compounds on the expression of the NPY or NPY receptor gene. Such analyses may reveal both quantitative and qualitative aspects of the expression pattern of the NPY or NPY receptor gene, including activation or inactivation of NPY or NPY receptor gene expression.

In one embodiment of such a detection scheme, cDNAs are synthesized from the RNAs of interest (e.g., by reverse transcription of the RNA molecule into cDNA). A sequence within the cDNA is then used as the template for a nucleic acid amplification reaction, such as a PCR amplification reaction, or the like. The nucleic acid reagents used as synthesis initiation reagents (e.g., primers) in the reverse transcription and nucleic acid amplification steps of this method are chosen from among NPY and NPY receptor nucleic acid reagents which are well known to those of skill in the art. The preferred lengths of such nucleic acid reagents are at least 9-30 nucleotides. For detection of the amplified product, the nucleic acid amplification may be performed using radioactively or non-radioactively labeled nucleotides. Alternatively, enough amplified product may be made such that the product may be visualized by standard ethidium bromide staining or by utilizing any other suitable nucleic acid staining method.

Additionally, it is possible to perform such NPY and NPY receptor gene expression assays “in situ”, ie., directly upon tissue sections (fixed and/or frozen) of patient tissue NPY obtained from biopsies or resections, such that no nucleic acid purification is necessary. Nucleic acid reagents which are well known to those of skill in the art may be used as probes and/or primers for such in situ procedures (See, for example, Nuovo, G. J., 1992, “PCR In situ Hybridization: Protocols And Applications”, Raven Press, NY).

Alternatively, if a sufficient quantity of the appropriate cells can be NPY obtained, standard Northern analysis can be performed to determine the level of mRNA expression of the NPY and NPY receptor gene.

5.2.2 Detection of NPY and NPY Receptor Gene Products

Antibodies directed against wild type or mutant NPY or NPY receptor gene products or conserved variants or peptide fragments thereof, which are discussed, above, in Section 5.1.1, may also be used as diagnostics and prognostics for bone disease, as described herein. Such diagnostic methods may be used to detect abnormalities in the level of NPY or NPY receptor gene expression, or abnormalities in the structure and/or temporal, tissue, cellular, or subcellular location of NPY or NPY receptor, and may be performed in vivo or in vitro, such as, for example, on biopsy tissue.

For example, antibodies directed to epitopes of the NPY receptor ECD or NPY can be used in vivo to detect the pattern and level of expression of the NPY or NPY receptor in the body. Such antibodies can be labeled, e.g., with a radio-opaque or other appropriate compound and injected into a subject in order to visualize binding to NPY or NPY receptor expressed in the body using methods such as X-rays, CAT-scans, or MRI. Labeled antibody fragments, e.g., the Fab or single chain antibody comprising the smallest portion of the antigen binding region, are preferred for this purpose to promote crossing the blood-brain barrier and permit labeling NPYs expressed in the brain.

Additionally, any NPY or NPY receptor fusion protein or NPY or NPY receptor conjugated protein whose presence can be detected, can be administered. For example, NPY or NPY receptor fusion or conjugated proteins labeled with a radio-opaque or other appropriate compound can be administered and visualized in vivo, as discussed, above for labeled antibodies. Further such fusion proteins can be utilized for in vitro diagnostic procedures.

Alternatively, immunoassays or fusion protein detection assays, as described above, can be utilized on biopsy and autopsy samples in vitro to permit assessment of the expression pattern of NPY or NPY receptor. Such assays are not confined to the use of antibodies that define any particular epitope of NPY or NPY receptor. The use of these labeled antibodies will yield useful information regarding translation and intracellular transport of NPY and NPY receptor to the cell surface, and can identify defects in processing.

The tissue or cell type to be analyzed will generally include those which are known, or suspected, to express the NPY or NPY receptor gene. The protein isolation methods employed herein may, for example, be such as those described in Harlow and Lane (Harlow, E. and Lane, D., 1988, “Antibodies: A Laboratory Manual”, Cold Spring Harbor Laboratory Press, Cold Spring Harbor, N.Y.), which is incorporated herein by reference in its entirety. The isolated cells can be derived from cell culture or from a patient. The analysis of cells taken from culture may be a necessary step in the assessment of cells that could be used as part of a cell-based gene therapy technique or, alternatively, to test the effect of compounds on the expression of the NPY or NPY receptor gene.

For example, antibodies, or fragments of antibodies, such as those described, above, in Section 5.1.1, useful in the present invention may be used to quantitatively or qualitatively detect the presence of NPY or NPY receptor gene products or conserved variants or peptide fragments thereof. This can be accomplished, for example, by immunofluorescence techniques employing a fluorescently labeled antibody (see below, this Section) coupled with light microscopic, flow cytometric, or fluorimetric detection. Such techniques are especially preferred if such NPY or NPY receptor gene products are expressed on the cell surface.

The antibodies (or fragments thereof) or NPY or NPY receptor fusion or conjugated proteins useful in the present invention may, additionally, be employed histologically, as in immunofluorescence, immunoelectron microscopy or non-immuno assays, for in situ detection of NPY and NPY receptor gene products or conserved variants or peptide fragments thereof, or for NPY receptor binding (in the case of labeled NPY receptor fusion protein).

In situ detection may be accomplished by removing a histological specimen from a patient, and applying thereto a labeled antibody or fusion protein of the present invention. The antibody (or fragment) or fusion protein is preferably applied by overlaying the labeled antibody (or fragment) onto a biological sample. Through the use of such a procedure, it is possible to determine not only the presence of the NPY or NPY receptor gene product, or conserved variants or peptide fragments, or NPY binding, but also its distribution in the examined tissue. Using the present invention, those of ordinary skill will readily perceive that any of a wide variety of histological methods (such as staining procedures) can be modified in order to achieve such in situ detection.

Immunoassays and non-immunoassays for NPY and NPY receptor gene products or conserved variants or peptide fragments thereof will typically comprise incubating a sample, such as a biological fluid (e.g., blood serum or cerebrospinal fluid), a tissue extract, freshly harvested cells, or lysates of cells which have been incubated in cell culture, in the presence of a detectably labeled antibody capable of identifying NPY or NPY receptor gene products or conserved variants or peptide fragments thereof, and detecting the bound antibody by any of a number of techniques well-known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled NPY or NPY receptor antibody or NPY or NPY receptor fusion protein. The solid phase support may then be washed with the buffer a second time to remove unbound antibody or fusion protein. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of NPY or NPY receptor antibody or NPY or NPY receptor fusion protein may be determined according to well known methods. Those skilled in the art will be able to determine operative and optimal assay conditions for each determination by employing routine experimentation.

With respect to antibodies, one of the ways in which the NPY or NPY receptor antibody can be detectably labeled is by linking the same to an enzyme and use in an enzyme immunoassay (EIA) (Voller, A., “The Enzyme Linked Immunosorbent Assay (ELISA)”, 1978, Diagnostic Horizons 2:1-7, Microbiological Associates Quarterly Publication, Walkersville, Md.); Voller, A. et al., 1978, J. Clin. Pathol. 31:507-520; Butler, J. E., 1981, Meth. Enzymol. 73:482-523; Maggio, E. (ed.), 1980, Enzyme Immunoassay, CRC Press, Boca Raton, Fla.,; Ishikawa, E. et al., (eds.), 1981, Enzyme Immunoassay, Kgaku Shoin, Tokyo). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alphaglycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by calorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect NPY or NPY receptor through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

5.3 Screening Assays for Compounds Useful in the Treatment, Diagnosis and Prevention of Bone Disease

The present invention also provides screening methods (e.g., assays) for the identification of compounds which affect bone disease. The invention further encompasses agonists and antagonists of NPY and NPY receptors, including small molecules, large molecules, and antibodies, as well as nucleotide sequences that can be used to inhibit NPY and NPY receptor gene expression (e.g., antisense and ribozyme molecules), and gene or regulatory sequence replacement constructs designed to enhance NPY or NPY receptor gene expression (e.g., expression constructs that place the NPY or NPY receptor.gene under the control of a strong promoter system). Such compounds may be used to treat bone diseases.

In particular, cellular and non-cellular assays are described that can be used to identify compounds that interact with NPY and NPY receptors, e.g., modulate the activity of NPY and NPY receptors and/or bind to the NPY receptor. The cell based assays can be used to identify compounds or compositions that affect the signal-transduction activity of NPY and NPY receptors, whether they bind to the NPY receptor or act on intracellular factors involved in the NPY signal transduction pathway. Such cell-based assays of the invention utilize cells, cell lines, or engineered cells or cell lines that express NPY or NPY receptors. The cells can be further engineered to incorporate a reporter molecule linked to the signal transduced by the activated NPY receptor to aid in the identification of compounds that modulate NPY and NPY receptors signaling activity.

The invention also encompasses the use of cell-based assays or cell-lysate assays (e.g., in vitro transcription or translation assays) to screen for compounds or compositions that modulate NPY and NPY receptor gene expression. To this end, constructs containing a reporter sequence linked to a regulatory element of the NPY or NPY receptor genes can be used in engineered cells, or in cell lysate extracts, to screen for compounds that modulate the expression of the reporter gene product at the level of transcription. For example, such assays could be used to identify compounds that modulate the expression or activity of transcription factors involved in NPY and NPY receptor gene expression, or to test the activity of triple helix polynucleotides. Alternatively, engineered cells or translation extracts can be used to screen for compounds (including antisense and ribozyme constructs) that modulate the translation of NPY and NPY receptors mRNA transcripts, and therefore, affect expression of the NPY receptor.

The following assays are designed to identify compounds that interact with (e.g., bind to) NPY or NPY receptor (including, but not limited to, the ECD or CD of NPY receptor), compounds that interact with (e.g., bind to) intracellular proteins that interact with NPY or NPY receptor (including, but not limited to, the TM and CD of NPY receptor), compounds that interfere with the interaction of NPY or NPY receptor with transmembrane or intracellular proteins involved in NPY receptor-mediated signal transduction, and to compounds which modulate the activity of NPY or NPY receptor gene expression or modulate the level of NPY or NPY receptor. Assays may additionally be utilized which identify compounds which bind to NPY or NPY receptor gene regulatory sequences (e.g., promoter sequences) and which may modulate NPY or NPY receptor gene expression.; See e.g., Platt, K. A., 1994, J. Biol. Chem. 269:28558-28562 Upon identification, compounds can further be tested for an ability to modulate NPY signalling in vitro or in vivo, and can still further be tested for an ability to modulate bone mass (that is, increase or decrease bone mass) and to treat a bone disease characterized by a decreased or an increased bone mass relative to a corresponding non-diseased bone.

Thus, in accordance with this aspects of the present invention, there is a method for identifying a compound to be tested for an ability to modulate (increase or decrease) bone mass in a mammal, comprising:

(a) contacting a test compound with a polypeptide; and

(b) determining whether the test compound binds the polypeptide, so that if the test compound binds the polypeptide, then a compound to be tested for an ability to modulate bone mass is identified, wherein the polypeptide is selected from the group consisting of a neuropeptide Y polypeptide and a neuropeptide Y receptor polypeptide.

Alternatively, there is a method for identifying a compound that modulates (increases or decreases) bone mass in a mammal, comprising:

(a) contacting test compounds with a polypeptide;

(b) identifying a test compound that binds the polypeptide; and

(c) administering the test compound in (b) to a non-human mammal, and determining whether the test compound modulates bone mass in the mammal relative to that of a corresponding bone in an untreated control non-human mammal, wherein the polypeptide is selected from the group consisting of a neuropeptide Y polypeptide and a neuropeptide Y receptor polypeptide, so that if the test compound modulates bone mass, then a compound that modulates bone mass in a mammal is identified.

In accordance with this, and other aspects of the present invention, a control non-human mammal, as used herein, is intended to mean a corresponding mammal that has not been administered the test compound

In accordance with yet another aspect of the present invention, there is a method for identifying a compound to be tested for an ability to modulate (increase or decrease) bone mass in a mammal, comprising:

(a) contacting a test compound with a neuropeptide Y polypeptide and a neuropeptide Y receptor polypeptide for a time sufficient to form neuropeptide Y/neuropeptide Y receptor complexes; and

(b) measuring neuropeptide Y/neuropeptide Y receptor complex level, so that if the level measured differs from that measured in the absence of the test compound, then a compound to be tested for an ability to modulate bone mass is identified.

In accordance with this, and other aspects of the present invention, neuropeptide Y/neuropeptide Y receptor complex formation can be measured by, for example, isolating the complex and determining the amount complex formation by various assays well known to those of skill in the art, e.g., Western Blot.

In accordance with another aspect of the present invention, there is a method for identifying a compound to be tested for an ability to decrease bone mass in a mammal, comprising:

(a) contacting a test compound with a cell which expresses a functional neuropeptide Y receptor; and

(b) determining whether the test compound activates the neuropeptide Y receptor, wherein if the compound activates the neuropeptide Y receptor a compound to be tested for an ability to decrease bone mass in a mammal is identified.

In accordance with this, and other aspects of the present invention, a functional neuropeptide Y receptor is a neuropeptide Y receptor which is capable of signal transduction following ligand binding to the active site of the receptor. Activation of the neuropeptide Y receptor, as used herein, is any increase in the activity (i.e., signal transduction) of the neuropeptide Y receptor.

In accordance with another aspect of the present invention, there is a method for identifying a compound that decreases bone mass in a mammal, comprising:

(a) contacting a test compound with a cell that expresses a functional neuropeptide Y receptor, and determining whether the test compound activates the neuropeptide Y receptor;

(b) administering a test compound identified in (a) as activating the neuropeptide Y receptor to a non-human animal, and determining whether the test compound decreases bone mass of the animal relative to that of a corresponding bone of a control-non-human animal, so that if the test compound decreases bone mass, then a compound that decreases bone mass in a mammal is identified.

In accordance with another aspect of the present invention, there is a method for identifying a compound to be tested for an ability to increase bone mass in a mammal, comprising:

(a) contacting a neuropeptide Y polypeptide and a test compound with a cell that expresses a functional neuropeptide Y receptor; and

(b) determining whether the test compound lowers activation of the neuropeptide Y receptor relative to that observed in the absence of the test compound; wherein a test compounds that lowers activation of the neuropeptide Y receptor is identified as a compound to be tested for an ability to increase bone mass in a mammal.

In accordance with yet another aspect of the present invention, there is a method for identifying a compound that increases bone mass in a mammal, comprising:

(a) contacting a neuropeptide Y polypeptide and a test compound with a cell that expresses a functional neuropeptide Y receptor, and determining whether the test compound decreases activation of the neuropeptide Y receptor;

(b) administering a test compound identified in (a) as decreasing neuropeptide Y receptor to a non-human animal, and determining whether the test compound increases bone mass of the animal relative to that of a corresponding bone of a control non-human animal, so that if the test compound increases bone mass, then a compound that increases bone mass in a mammal is identified.

In accordance with yet another aspect of the invention, there is a method in which activation of a neuropeptide Y receptor is determined by measuring levels of inositol phosphate or extracellular signal-regulated kinase, which are downstream effectors of neuropeptide Y signaling in its target cells. Inositol phosphate is increased and extracellular signal-regulated kinase is activated following activation of the neuropeptide Y receptor by neuropeptide Y.

The compounds which may be screened in accordance with the invention include, but are not limited to, peptides, antibodies and fragments thereof, and other organic compounds (e.g., peptidomimetics) that bind to NPY or NPY receptor and either mimic the activity triggered by the natural ligand (i.e., agonists) or inhibit the activity triggered by the natural ligand (i.e., antagonists); as well as peptides, antibodies or fragments thereof, and other organic compounds that mimic the ECD of the NPY receptor (or a portion thereof) and bind to and “neutralize” natural ligand. Additional compounds which may be screened in accordance with the invention include, but are not limited to, compounds which interact with NPY and prevent the transport of NPY across the blood-brain barrier, thereby preventing NPY from activating the NPY receptor.

Such compounds may include, but are not limited to, peptides such as, for example, soluble peptides, including but not limited to members of random peptide libraries; (see, e.g., Lam, K. S. et al., 1991, Nature 354:82-84; Houghten, R. et al., 1991, Nature 354:84-86), and combinatorial chemistry-derived molecular library made of D- and/or L-configuration amino acids, phosphopeptides (including, but not limited to, members of random or partially degenerate, directed phosphopeptide libraries; see, e.g., Songyang, Z. et al., 1993, Cell 72:767-778), antibodies (including, but not limited to, polyclonal, monoclonal, human, humanized, anti-idiotypic, chimeric or single chain antibodies, and FAb, F(ab′)₂ and FAb expression library fragments, and epitope-binding fragments thereof), and small organic or inorganic molecules.

Other compounds which can be screened in accordance with the invention include, but are not limited to, small organic molecules that are able to cross the blood-brain barrier, gain entry into an appropriate cell and affect the expression of the NPY or NPY receptor gene or some other gene involved in the NPY receptor signal transduction pathway (e.g., by interacting with the regulatory region or transcription factors involved in gene expression); or such compounds that affect the activity of the NPY receptor (e.g., by inhibiting or enhancing the enzymatic activity of the CD) or the activity of some other intracellular factor involved in the NPY receptor signal transduction pathway.

Computer modeling and searching technologies permit identification of compounds, or the improvement of already identified compounds, that can modulate NPY or NPY receptor expression or activity. Having identified such a compound or composition, the active sites or regions are identified. Such active sites might typically be ligand binding sites, such as the interaction domains of NPY with NPY receptor itself. The active site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found. Next, the three dimensional geometric structure of the active site is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures may be measured with a complexed ligand, natural or artificial, which may increase the accuracy of the active site structure determined.

If an incomplete or insufficiently accurate structure is determined, the methods of computer based numerical modeling can be used to complete the structure or improve its accuracy. Any recognized modeling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site, either experimentally, by modeling, or by a combination, candidate modulating compounds can be identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential NPY or NPY receptor modulating compounds.

Alternatively, these methods can be used to identify improved modulating compounds from an already known modulating compound or ligand. The composition of the known compound can be modified and the structural effects of modification can be determined using the experimental and computer modeling methods described above applied to the new composition. The altered structure is then compared to the active site structure of the compound to determine if an improved fit or interaction results. In this manner, systematic variations in composition, such as by varying side groups, can be quickly evaluated to obtain modified modulating compounds or ligands of improved specificity or activity.

Further experimental and computer modeling methods useful to identify modulating compounds based upon identification of the active sites of NPY, NPY receptor, and related transduction and transcription factors will be apparent to those of skill in the art.

Examples of molecular modeling systems are the CHARMm and QUANTA programs (Polygen Corporation, Waltham, Mass.). CHARMm performs the energy minimization and molecular dynamics functions. QUANTA performs the construction, graphic modelling and analysis of molecular structure. QUANTA allows interactive construction, modification, visualization, and analysis of the behavior of molecules with each other.

A number of articles review computer modeling of drugs interactive with specific-proteins, such as Rotivinen, et al., 1988, Acta Pharmaceutical Fennica 97:159-166; Ripka, New Scientist 54-57 (Jun. 16, 1988); McKinaly and Rossmann, 1989, Annu. Rev. Pharmacol. Toxiciol. 29:111-122; Perry and Davies, OSAR: Quantitative Structure-Activity Relationships in Drug Design pp. 189-193 (Alan R. Liss, Inc. 1989); Lewis and Dean, 1989 Proc. R. Soc. Lond. 236:125-140 and 141-162; and, with respect to a model receptor for nucleic acid components, Askew, et al., 1989, J. Am. Chem. Soc. 111:1082-1090. Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, they can be adapted to design of drugs specific to regions of DNA or RNA, once that region is identified.

Although described above with reference to design and generation of compounds which could alter binding, one could also screen libraries of known compounds, including natural products or synthetic chemicals, and biologically active materials, including proteins, for compounds which are inhibitors or activators.

Compounds identified via assays such as those described herein may be useful, for example, in elaborating the biological function of the NPY or NPY receptor gene product, and for ameliorating bone diseases. Assays for testing the effectiveness of compounds, identified by, for example, techniques such as those described in Section 5.3.1 through 5.3.3,are discussed, below, in Section 5.3.4.

5.3.1 In vitro Screening Assays for Compounds that Bind to NPY and NPY Receptor

In vitro systems may be designed to identify compounds capable of interacting with (e.g., binding to) NPY and NPY receptor (including, but not limited to, the ECD or CD of NPY receptor). Compounds identified may be useful, for example, in modulating the activity of wild type and/or mutant NPY or NPY receptor gene products; may be useful in elaborating the biological function of NPY or NPY receptor; may be utilized in screens for identifying compounds that disrupt normal NPY and NPY receptor interactions; or may in themselves disrupt such interactions.

The principle of the assays used to identify compounds that bind to NPY or NPY receptor involves preparing a reaction mixture of NPY or NPY receptor and the test compound under conditions and for a time sufficient to allow the two components to interact and bind, thus forming a complex which can be removed and/or detected in the reaction mixture. The NPY or NPY receptor species used can vary depending upon the goal of the screening assay. For example, where agonists of the natural ligand are sought, the full length NPY receptor, or a soluble truncated NPY receptor, e.g., in which the TM and/or CD is deleted from the molecule, a peptide corresponding to the ECD or a fusion protein containing the NPY receptor ECD fused to a protein or polypeptide that affords advantages in the assay system (e.g., labeling, isolation of the resulting complex, etc.) can be utilized. Where compounds that interact with the NPY receptor cytoplasmic domain are sought to be identified, peptides corresponding to the NPY receptor CD and fusion proteins containing the NPY receptor CD can be used. In addition, where compounds which will prevent NPY entry across the blood-brain barrier are sought, NPY, or soluble forms of NPY, can be used.

The screening assays can be conducted in a variety of ways. For example, one method to conduct such an assay would involve anchoring the NPY or NPY receptor protein, polypeptide, peptide or fusion protein or the test substance onto a solid phase and detecting NPY or NPY receptor/test compound complexes anchored on the solid phase at the end of the reaction. In one embodiment of such a method, the NPY or NPY receptor reactant may be anchored onto a solid surface, and the test compound, which is not anchored, may be labeled, either directly or indirectly.

In practice, microtiter plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for NPY or NPY receptor protein, polypeptide, peptide or fusion protein or the test compound to anchor any complexes formed in solution, and a labeled antibody specific for the other component of the possible complex to detect anchored complexes.

Alternatively, cell-based assays can be used to identify compounds that interact with NPY or NPY receptor. To this end, cell lines that express NPY or NPY receptor, or cell lines (e.g., PC 12 cells, COS cells, CHO cells, fibroblasts, etc.) that have been genetically engineered to express NPY or NPY receptor (e.g., by transfection or transduction of NPY or NPY receptor DNA) can be used. Interaction of the test compound with, for example, the ECD of NPY receptor expressed by the host cell can be determined by comparison or competition with native NPY.

5.3.2 Assays for Proteins that Interact with NPY and NPY Receptor

Any method suitable for detecting protein-protein interactions may be employed for identifying transmembrane proteins or intracellular proteins that interact with NPY or NPY receptor. Among the traditional methods which may be employed are co-immunoprecipitation, crosslinking and co-purification through gradients or chromatographic columns of cell lysates or proteins obtained from cell lysates and NPY or NPY receptor to identify proteins in the lysate that interact with NPY or NPY receptor. For these assays, the NPY or NPY receptor component used can be full length, a soluble derivative lacking the membrane-anchoring region (e.g., a truncated NPY receptor in which the TM is deleted resulting in a truncated molecule containing the ECD fused to the CD), a peptide corresponding to the CD or a fusion protein containing NPY or the CD of NPY receptor. Once isolated, such an intracellular protein can be identified and can, in turn, be used in conjunction with standard techniques, to identify proteins with which it interacts. For example, at least a portion of the amino acid sequence of an intracellular protein which interacts with NPY or NPY receptor can be ascertained using techniques well known to those of skill in the art, such as via the Edman degradation technique. (See, e.g., Creighton, 1983, “Proteins: Structures and Molecular Principles”, W. H. Freeman & Co., N.Y., pp.34-49). The amino acid sequence obtained may be used as a guide for the generation of oligonucleotide mixtures that can be used to screen for gene sequences encoding such intracellular proteins. Screening may be accomplished, for example, by standard hybridization or PCR techniques. Techniques for the generation of oligonucleotide mixtures and the screening are well-known. (See, e.g., Ausubel, supra., and PCR Protocols: A Guide to Methods and Applications, 1990, Innis, M. et al., eds. Academic Press, Inc., New York).

Additionally, methods may be employed which result in the simultaneous identification of genes which encode the transmembrane or intracellular proteins interacting with NPY receptor or NPY. These methods include, for example, probing expression libraries in a manner similar to the well known technique of antibody probing of λgt11 libraries, using labeled NPY or NPY receptor protein, or an NPY or NPY receptor polypeptide, peptide or fusion protein, e.g., an NPY or NPY receptor polypeptide or an NPY or NPY receptor domain fused to a marker (e.g., an enzyme, fluor, luminescent protein, or dye), or an Ig-Fc domain.

One method which detects protein interactions in vivo, the two-hybrid system, is described in detail for illustration only and not by way of limitation. One version of this system has been described (Chien et al., 1991, Proc. Natl. Acad. Sci. USA, 88:9578-9582) and is commercially available from Clontech (Palo Alto, Calif.).

Briefly, utilizing such a system, plasmids are constructed that encode two hybrid proteins: one plasmid consists of nucleotides encoding the DNA-binding domain of a transcription activator protein fused to an NPY or NPY receptor nucleotide sequence encoding NPY or NPY receptor, an NPY or NPY receptor polypeptide, peptide or fusion protein, and the other plasmid consists of nucleotides encoding the transcription activator protein's activation domain fused to a cDNA encoding an unknown protein which has been recombined into this plasmid as part of a cDNA library. The DNA-binding domain fusion plasmid and the cDNA library are transformed into a strain of the yeast Saccharomyces cerevisiae that contains a reporter gene (e.g., HBS or lacZ) whose regulatory region contains the transcription activator's binding site. Either hybrid protein alone cannot activate transcription of the reporter gene: the DNA-binding domain hybrid cannot because it does not provide activation function and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology may be used to screen activation domain libraries for proteins that interact with the “bait” gene product. By way of example, and not by way of limitation, NPY or NPY receptor may be used as the bait gene product. Total genomic or cDNA sequences are fused to the DNA encoding an activation domain. This library and a plasmid encoding a hybrid of a bait NPY or NPY receptor gene product fused to the DNA-binding domain are cotransformed into a yeast reporter strain, and the resulting transformants are screened for those that express the reporter gene. For example, and not by way of limitation, a bait NPY or NPY receptor gene sequence, such as the open reading frame of NPY or NPY receptor (or a domain of NPY receptor), can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with bait NPY or NPY receptor gene product are to be detected can be made using methods routinely practiced in the art. According to the particular system described herein, for example, the cDNA fragments can be inserted into a vector such that they are translationally fused to the transcriptional activation domain of GAL4. This library can be co-transformed along with the bait NPY or NPY receptor gene-GAL4 fusion plasmid into a yeast strain which contains a lacZ gene driven by a promoter which contains GAL4 activation sequence. A cDNA encoded protein, fused to GAL4 transcriptional activation domain, that interacts with bait NPY or NPY receptor gene product will reconstitute an active GALA protein and thereby drive expression of the HIS3 gene. Colonies which express HIS3 can be detected by their growth on petri dishes containing semi-solid agar based media lacking histidine. The cDNA can then be purified from these strains, and used to produce and isolate the bait NPY or NPY receptor gene-interacting protein using techniques routinely practiced in the art.

5.3.3 Assays for Compounds that Interfere with NPY and NPY Receptor/Intracellular or NPY Receptor/Transmembrane Macromolecule Interactions

The macromolecules that interact with NPY or NPY receptor are referred to, for purposes of this discussion, as “binding partners”. These binding partners are likely to be involved in the NPY receptor signal transduction pathway, and therefore, in the role of NPY or NPY receptor in regulation of bone disorders. Therefore, it is desirable to identify compounds that interfere with or disrupt the interaction of such binding partners with NPY which may be useful in regulating the activity of the NPY receptor and control bone disorders associated with NPY receptor activity.

The basic principle of the assay systems used to identify compounds that interfere with the interaction between NPY or NPY receptor and their binding partner or partners involves preparing a reaction mixture containing NPY or NPY receptor protein, polypeptide, peptide or fusion protein as described above, and the binding partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of the NPY or NPY receptor moiety and its binding partner. Control reaction mixtures are incubated without the test compound or with a placebo. The formation of any complexes between the NPY or NPY receptor moiety and the binding partner is then detected. The formation of a complex in the control reaction, but not in the reaction mixture containing the test compound, indicates that the compound interferes with the interaction of NPY or NPY receptor and the interactive binding partner. Additionally, complex formation within reaction mixtures containing the test compound and normal NPY or NPY receptor protein may also be compared to complex formation within reaction mixtures containing the test compound and a mutant NPY or NPY receptor. This comparison may be important in those cases wherein it is desirable to identify compounds that disrupt interactions of mutant but not normal NPY or NPY receptors.

The assay for compounds that interfere with the interaction of NPY or NPY receptor and binding partners can be conducted in a heterogeneous or homogeneous format. Heterogeneous assays involve anchoring either the NPY or NPY receptor moiety product or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction by competition can be identified by conducting the reaction in the presence of the test substance; i.e., by adding the test substance to the reaction mixture prior to or simultaneously with the NPY or NPY receptor moiety and interactive binding partner. Alternatively, test compounds that disrupt preformed complexes, e.g. compounds with higher binding constants that displace one of the components from the complex, can be tested by adding the test compound to the reaction mixture after complexes have been formed. The various formats are described briefly below.

In a heterogeneous assay system, either the NPY or NPY receptor moiety or the interactive binding partner, is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of the NPY or NPY receptor gene product or binding partner and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds which inhibit complex or which disrupt preformed complexes can be identified.

In an alternate embodiment of the invention, a homogeneous assay can be used. In this approach, a preformed complex of the NPY or NPY receptor moiety and the interactive binding partner is prepared in which either NPY or NPY receptor or its binding partners is labeled, but the signal generated by the label is quenched due to formation of the complex (see, e.g., U.S. Pat. No. 4,109,496 by Rubenstein which utilizes this approach for immunoassays). The addition of a test substance that competes with and displaces one of the species from the preformed complex will result in the generation of a signal above background. In this way, test substances which disrupt NPY or NPY receptor/intracellular binding partner interaction can be identified.

In a particular embodiment, an NPY or NPY receptor fusion can be prepared for immobilization. For example, the NPY or NPY receptor or a peptide fragment, e.g., corresponding to the NPY receptor CD, can be fused to a glutathione-S-transferase (GST) gene using a fusion vector, such as pGEX-5X-1, in such a manner that its binding activity is maintained in the resulting fusion protein. The interactive binding partner can be purified and used to raise a monoclonal antibody, using methods routinely practiced in the art. This antibody can be labeled with the radioactive isotope ¹²⁵I, for example, by methods routinely practiced in the art. In a heterogeneous assay, e.g., the GST-NPY receptor fusion protein can be anchored to glutathione-agarose beads. The interactive binding partner can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between the NPY or NPY receptor gene product and the interactive binding partner can be detected by measuring the amount of radioactivity that remains associated with the glutathione-agarose beads. A successful inhibition of the interaction by the test compound will result in a decrease in measured radioactivity.

Alternatively, the GST-NPY/NPY receptor fusion protein and the interactive binding partner can be mixed together in liquid in the absence of the solid glutathione-agarose beads. The test compound can be added either during or after the species are allowed to interact. This mixture can then be added to the glutathione-agarose beads and unbound material is washed away. Again the extent of inhibition of the NPY or NPY receptor/binding partner interaction can be detected by adding the labeled antibody and measuring the radioactivity associated with the beads.

In another embodiment of the invention, these same techniques can be employed using peptide fragments that correspond to the binding domains of NPY or NPY receptor and/or the interactive or binding partner (in cases where the binding partner is a protein), in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interactive binding. Alternatively, one protein can be anchored to a solid surface using methods described above, and allowed to interact with and bind to its labeled binding partner, which has been treated with a proteolytic enzyme, such as trypsin. After washing, a short, labeled peptide comprising the binding domain may remain associated with the solid material, which can be isolated and identified by amino acid sequencing. Also, once the gene coding for the intracellular binding partner is obtained, short gene segments can be engineered to express peptide fragments of the protein, which can then be tested for binding activity and purified or synthesized.

For example, and not by way of limitation, an NPY or NPY receptor gene product can be anchored to a solid material as described, above, by making a GST-NPY or —NPY receptor fusion protein and allowing it to bind to glutathione agarose beads. The interactive binding partner can be labeled with a radioactive isotope, such as ³⁵S, and cleaved with a proteolytic enzyme such as trypsin. Cleavage products can then be added to the anchored GST-NPY or —NPY receptor fusion protein and allowed to bind. After washing away unbound peptides, labeled bound material, representing the intracellular binding partner binding domain, can be eluted, purified, and analyzed for amino acid sequence by well-known methods. Peptides so identified can be produced synthetically or fused to appropriate facilitative proteins using recombinant DNA technology.

5.3.4 Assays for Identification of Compounds that Ameliorate Bone Disease

Compounds, including, but not limited to, compounds identified via assay techniques such as those described, above, in Sections 5.3.1 through 5.3.3, can be tested for the ability to treat bone disease and ameliorate bone disease symptoms. The assays described above can identify compounds which affect NPY or NPY receptor activity (e.g., NPY receptor agonists or antagonists), and compounds that bind to the natural ligand of the NPY receptor and neutralize ligand activity; or compounds that affect NPY or NPY receptor gene activity (by affecting NPY or NPY receptor gene expression, including molecules, e.g., proteins or small organic molecules, that affect or interfere with splicing events so that expression of the full length or the truncated form of the NPY or NPY receptor can be modulated). However, it should be noted that the assays described can also identify compounds that modulate NPY or NPY receptor signal transduction (e.g., compounds which affect downstream signaling events, such as inhibitors or enhancers of tyrosine kinase or phosphatase activities which participate in transducing the signal activated by NPY binding to the NPY receptor). Alternatively, the assays described can also identify compounds which modulate the entry of NPY through the blood-brain barrier. The identification and use of such compounds which affect another step in the NPY or NPY receptor signal transduction pathway in which the NPY or NPY receptor gene and/or gene product is involved and, by affecting this same pathway may modulate the effect of NPY or NPY receptor on the development of bone disorders are within the scope of the invention. Such compounds can be used as part of a therapeutic method for the treatment of bone disease.

Cell-based systems can be used to identify compounds which may act to ameliorate bone disease. Such cell systems can include, for example, recombinant or non-recombinant cells, such as cell lines, which express the NPY or NPY receptor gene. For example, PC-12 cell line can be utilized. In addition, expression host cells (e.g., COS cells, CHO cells, fibroblasts) genetically engineered to express a functional NPY or NPY receptor and to respond to activation by the natural NPY ligand, e.g., as measured by a chemical or phenotypic change, induction of another host cell gene, change in ion flux (e.g., Ca⁺⁺), tyrosine phosphorylation of host cell proteins, ERK activation, inositol phosphate levels etc., can be used as an end point in the assay.

In utilizing such cell systems, cells may be exposed to a compound suspected of exhibiting an ability to ameliorate bone disorders, at a sufficient concentration and for a time sufficient to elicit such an amelioration of bone disorders in the exposed cells. After exposure, the cells can be assayed to measure alterations in the expression of the NPY or NPY receptor gene, e.g., by assaying cell lysates for NPY or NPY receptor mRNA transcripts (e.g., by Northern analysis) or for NPY or NPY receptor protein expressed in the cell; compounds which regulate or modulate expression of the NPY or NPY receptor gene are good candidates as therapeutics. Alternatively, the cells are examined to determine whether one or more bone disorder-like cellular phenotypes has been altered to resemble a more normal or more wild type, non-bone disorder phenotype, or a phenotype more likely to produce a lower incidence or severity of disorder symptoms. Still further, the expression and/or activity of components of the signal transduction pathway of which NPY receptor is a part, or the activity of the NPY receptor signal transduction pathway itself can be assayed.

For example, after exposure, the cell lysates can be assayed for the presence of tyrosine phosphorylation of host cell proteins, as compared to lysates derived from unexposed control cells. The ability of a test compound to inhibit tyrosine phosphorylation of host cell proteins in these assay systems indicates that the test compound inhibits signal transduction initiated by NPY receptor activation. The cell lysates can be readily assayed using a Western blot format; i.e., the host cell proteins are resolved by gel electrophoresis, transferred and probed using a anti-phosphotyrosine detection antibody (e.g., an anti-phosphotyrosine antibody labeled with a signal generating compound, such as radiolabel, fluor, enzyme, etc.) (See, e.g., Glenney et al., 1988, J. Immunol. Methods 109:277-285; Frackelton et al., 1983, Mol. Cell. Biol. 3:1343-1352). Alternatively, an ELISA format could be used in which a particular host cell protein involved in the NPY receptor signal transduction pathway is immobilized using an anchoring antibody specific for the target host cell protein, and the presence or absence of phosphotyrosine on the immobilized host cell protein is detected using a labeled anti-phosphotyrosine antibody. (See, King et al., 1993, Life Sciences 53:1465-1472). In yet another approach, ion flux, such as calcium ion flux, can be measured as an end point for NPY receptor stimulated signal transduction.

In addition, animal-based bone disorder systems, such as can be generated via the transgenic animal techniques described above, may be used to identify compounds capable of ameliorating bone disorder-like symptoms. Such animal models may be used as test substrates for the identification of drugs, pharmaceuticals, therapies and interventions which may be effective in treating such disorders. For example, animal models may be exposed to a compound suspected of exhibiting an ability to ameliorate bone disorder symptoms, at a sufficient concentration and for a time sufficient to elicit such an amelioration of bone disorder symptoms in the exposed animals. The response of the animals to the exposure may be monitored by assessing the reversal of disorders associated with bone disorders such as osteoporosis. With regard to intervention, any treatments which reverse any aspect of bone disorder-like symptoms should be considered as candidates for human bone disorder therapeutic intervention. Dosages of test agents may be determined by deriving dose-response curves, as discussed below.

5.4 Compounds that Modulate NPY or NPY-R Expression or Activity

Compounds that interact with (e.g., bind to) NPY or NPY-R (including, but not limited to, the ECD or CD of NPY-R), compounds that interact with (e.g., bind to) intracellular proteins that interact with NPY or NPY-R (including, but not limited to, the TM and CD of NPY-R), compounds that interfere with the interaction of NPY or NPY-R with transmembrane or intracellular proteins involved in NPY-R-mediated signal transduction, and compounds which modulate the activity of NPY or NPY-R gene expression or modulate the level of NPY or NPY-R are capable of modulating levels of bone mass. More specifically, compounds which decrease the levels of NPY or NPY-R, inhibit the transport of NPY across the blood-brain barrier or inhibit binding of NPY to the NPY-R would cause an increase in bone mass.

Examples of such compounds are NPY and NPY receptor agonists and antagonists. NPY receptor antagonist, as used herein, refers to a factor which neutralizes or impedes or otherwise reduces the action or effect of a NPY receptor. Such antagonists can include compounds that bind NPY or that bind NPY receptor. Such antagonists can also include compounds that neutralize, impede or otherwise reduce NPY receptor output, that is, intracellular steps in the NPY signaling pathway following binding of NPY to the NPY receptor, i.e., downstream events that affect NPY/NPY receptor signaling, that do not occur at the receptor/ligand interaction level. NPY receptor antagonists may include, but are not limited to proteins, antibodies, small organic molecules or carbohydrates, such as, for example, acetylphenol compounds, antibodies which specifically bind NPY, antibodies which specifically bind NPY receptor, and compounds that comprise soluble NPY receptor polypeptide sequences.

For example, NPY antagonists also include agents, or drugs, which decrease, inhibit, block, abrogate or interfere with binding of NPY to its receptors or extracellular domains thereof, agents which decrease, inhibit, block, abrogate or interfere with NPY production or activation; agents which are antagonists of signals that drive NPY production or synthesis, and agents which prohibit NPY from reaching its receptor, e.g., prohibit NPY from crossing the blood-brain barrier. Such an agent can be any organic molecule that inhibits or prevents the interaction of NPY with its receptor, or NPY production.

NPY receptor agonist, as used herein, refers to a factor which activates, induces or otherwise increases the action or effect of a NPY receptor. Such agonists can include compounds that bind NPY or that bind NPY receptor. Additional NPY agonists and analogs include those described in U.S. Pat. No. 5,328,899. Such antagonists can also include compounds that activate, induce or otherwise increase NPY receptor output, that is, intracellular steps in the NPY signaling pathway following binding of NPY to the NPY receptor, i.e., downstream events that affect NPY/NPY receptor signaling, that do not occur at the receptor/ligand interaction level. NPY receptor agonists may include, but are not limited to proteins, antibodies, small organic molecules or carbohydrates, such as, for example, NPY, NPY analogs, and antibodies which specifically bind and activate NPY. NPY antagonists include, but are not limited to, anti-NPY antibodies, receptor molecules and derivatives which bind specifically to NPY and prevent NPY from binding to its cognate receptor.

Numerous NPY antagonists have been described. For example, U.S. Pat. No. 5,972,888 describes various compounds which act as NPY antagonists, including, but not limited to, derivatives of naphthalenes, benzofuran, benzothiophenes and indoles; raloxifene; 3-(4-methoxyphenyl)-4-[4-(2-pyrrolidin-1-ylethoxy)benzoyl-1,2-dihydronaphthalene, citrate salt; 3-phenyl-4-[4-(2-pyrrolidin-1-ylethoxy)benzoyl]-7-methoxy-1,2-dihydronaphthalene; 3-phenyl-4-[4-(2-pyrrolidin-1-ylethoxy)benzoyl]-1,2-dihydronaphthalene; 1-[4-(2-pyrrolidin-1-ylethoxy)benzoyl]-2-phenylnaphthalene, citrate salt; 3-(4-methoxyphenyl)-4-[4-[2-(piperidin-1-yl)ethoxy]benzoyl]-1,2-dihydronaphthalene, citrate salt; 3-(4-methoxyphenyl)-4-[4-(2-dimethylaminoethoxy)benzoyl]-1,2-dihydronaphthalene, citrate salt; 3-(4-hydroxyphenyl)-4-[4-[2-(pyrrolidin-1-yl)ethoxy]benzoyl]-1,2-dihydronaphthalene, mesylate salt; 3-(4-methoxyphenyl)-4-[4-[2-(hexamethyleneimin-1-yl)benzoyl]-1,2-dihydronaphthalene, mesylate salt; 3-(4-methoxyphenyl)-4-[4-[2-(piperidin-1-yl)ethoxy]benzoyl]-1,2-dihydronaphthalene, mesylate salt; 3-(4-methoxyphenyl)-4-(4-diethylaminoethoxybenzoyl)-1,2-dihydronaphthalene, mesylate salt; 3-(4-methoxyphenyl)-4-(4-diisopropylaminoethoxybenzoyl)-1,2-dihydronaphthalene, mesylate salt; 3-hydroxy-4-[4-[2-(pyrrolidin-1-yl)ethoxy]benzoyl]-1,2-dihydronaphthalene, sodium salt; 2-(4-methoxyphenyl)-1-[4-[2-(pyrrolidin-1-yl)ethoxy]benzoyl]naphthalene, mesylate salt; 3-(4-methoxyphenyl)-4-[4-[2-(piperidin-1-yl)ethoxy]benzoyl]-7-methoxy-1,2-d ihydronaphthalene, mesylate salt; 3-(4-methoxyphenyl)-4-[4-(2-dimethylaminoethoxy)benzoyl]-1,2-dihydronaphthalene, 2-hydroxy-1,2,3-propanetricarboxylic acid salt; 3-(4-methoxyphenyl)-4-[4-[2-(N-methyl-1-pyrrolidinium)ethoxy]benzoyl]-1,2-dihydronaphthalene, iodide salt; and 3-(4-methoxyphenyl)-4-[4-[2-(pyrrolidin-1-yl)ethoxy]benzoyl]-1,2-dihydronaphthalene, mesylate salt.

Similarly, numerous NPY-R antagonists have been identified. Examples of such antagonists include, but are not limited to, α-alkoxy and α-thioalkoxyamide compositions (See, e.g., U.S. Pat. No. 5,939,462); dihydropyridine based compounds (See, e.g., U.S. Pat. Nos. 5,554,621, 6,001,836, 5,668,151 and 5,635,503); substituted benzylamine derivatives (See, e.g., U.S. Pat. Nos. 5,985,873, 5,962,455 and 5,900,415); dihydropyrimidone derivatives (See, e.g., U.S. Pat. No. 5,889,016); naphthimidazolyl derivatives (See, e.g., U.S. Pat. No. 5,776,931); dimesylate salts (See, e.g., U.S. Pat. No. 5,914,329); and substituted benzofurans, benzothiophenes or indoles (See, e.g., U.S. Pat. No. 5,663,192). Additional NPY-R antagonists are disclosed in U.S. Pat. Nos. 5,567,714, 5,504,094, 5,670,482, 5,989,920, and 5,827,853, 5,985,616.

5.5. Methods for the Treatment or Prevention of Bone Disease

Bone diseases which can be treated and/or prevented in accordance with the present invention include bone diseases characterized by a decreased bone mass relative to that of corresponding non-diseased bone, including, but not limited to osteoporosis, osteopenia and Paget's disease. Bone diseases which can be treated and/or prevented in accordance with the present invention also include bone diseases characterized by an increased bone mass relative to that of corresponding non-diseased bone, including, but not limited to osteopetrosis, osteosclerosis and osteochondrosis.

In one aspect of the invention is a method of treating a bone disease comprising: administering to a mammal in need of said treatment a therapeutically effective amount of a compound that lowers neuropeptide Y level in blood serum, wherein the bone disease is characterized by a decreased bone mass relative to that of corresponding non-diseased bone. Specific embodiments of some of these compounds and methods include, but are not limited to ones that inhibit or lower neuropeptide Y synthesis or increase neuropeptide Y breakdown. Among such compounds are antisense, ribozyme or triple helix sequences of a neuropeptide Y-encoding polypeptide.

In accordance with another aspect of the present invention, there is a method of treating a bone disease comprising: administering to a mammal in need of said treatment a therapeutically effective amount of a compound that lowers neuropeptide Y level in cerebrospinal fluid, wherein the bone disease is characterized by a decreased bone mass relative to that of corresponding non-diseased bone. Specific embodiments of some of these compounds and methods include, but are not limited to ones that inhibit or lower neuropeptide Y synthesis or increase neuropeptide Y breakdown, and compounds that bind neuropeptide Y in blood.

Particular embodiments of the methods of the invention include, for example, a method of treating a bone disease comprising: administering to a mammal in need of said treatment a therapeutically effective amount of a compound, wherein the bone disease is characterized by a decreased bone mass relative to that of corresponding non-diseased bone, and wherein the compound is selected from the group consisting of compounds which bind neuropeptide Y in blood, including, but not limited to such compounds as an antibody which specifically binds neuropeptide Y, and a soluble neuropeptide Y receptor polypeptide.

In accordance with another aspect of the present invention, there is a method of treating a bone disease comprising: administering to a mammal in need of said treatment a therapeutically effective amount of a compound that lowers the level of ERK activation and inositol phosphate formation, wherein the bone disease is characterized by a decreased bone mass relative to that of corresponding non-diseased bone. Specific embodiments of some of these compounds and methods include, but are not limited to ones that inhibit or lower neuropeptide Y synthesis or increase neuropeptide Y breakdown, compounds that bind neuropeptide Y in blood, and neuropeptide Y receptor antagonist compounds, antibodies which specifically bind neuropeptide Y, antibodies which specifically bind neuropeptide Y receptor, and compounds that comprise soluble neuropeptide Y receptor polypeptide sequences.

A compound that lowers neuropeptide Y levels in blood serum or in cerebrospinal fluid is one that lowers neuropeptide Y levels in the following assay: contacting the compound with a cell from a neuropeptide Y expressing cell line, preferably a PC 12 cell line, and determining whether NPY expression and/or synthesis is lowered relative to the level exhibited by the cell line in the absence of the compound. Standard assays such as Northern Blot can be used to determine levels of NPY expression and Western Blot can be used to determine levels of NPY synthesis. An alternate assay comprises comparing the level of NPY in a mammal being treated for bone disease before and after administration of the compound, such that, if the level of NPY decreases, the compound is one that lowers NPY levels. Likewise, a compound that increases neuropeptide Y levels in blood serum or in cerebrospinal fluid is one that increases neuropeptide Y levels via such assays.

A compound that lowers the level of ERK activation and inositol phosphate formation, which are downstream effectors of NPY signaling in its target cells (Keffel et al., 1999, J Pharmacol Exp Ther, 291:1172-1178; Zheng et al., Biochem Biophys Res Comm, 239:287-290), is one that lowers the level of activated ERK and inositol phosphate in the following assay: contacting a NPY polypeptide and the compound with a cell that expresses a functional NPY receptor and determining the level of activated ERK and inositol phosphate in the cell. To determine the level of activated ERK and inositol phosphate, the cells can, for example, be lysed and an appropriate analysis (e.g., Western Blot) can be performed. If the level of activated ERK and inositol phosphate decreases relative to the level exhibited by the cell line in the absence of the compound, the compound is one that lowers the level of activated ERK and inositol phosphate. Likewise, a compound that increases the level of ERK activation and inositol phosphate formation polypeptide in blood serum or in cerebrospinal fluid is one that increases neuropeptide Y levels via such assays.

A compound is said to be administered in a “therapeutically effective amount” if the amount administered results in a desired change in the physiology of a recipient mammal, e.g., results in an increase or decrease in bone mass relative to that of a corresponding bone in the diseased state; that is, results in treatment; i.e., modulates bone mass to more closely resemble that of corresponding non-diseased bone (that is a corresponding bone of the same type, e.g., long, vertebral, etc.) in a non-diseased state. With respect to these methods, a corresponding non-diseased bone refers to a bone of the same type as the bone being treated (e.g., a corresponding vertebral or long bone), and bone mass is measured using standard techniques well known to those of skill in the art and described above, and include, for example, X-ray, DEXA and classical histological assessments and measurements of bone mass.

Among the compounds that can be utilized as part of the methods presented herein are those described, for example, in the sections and teached presented herein, as well as compounds identified via techniques such as those described in the sections and teaching presented herein.

Particular techniques and methods that can be utilized as part of the therapeutic and preventative methods of the invention are presented in detail below.

5.5.1. Inhibition of NPY or NPY-R Expression, Levels or Activity to Treat Bone Disease by Increasing Bone Mass

Any method which neutralizes, slows or inhibits NPY or NPY-R expression (either transcription or translation), levels, or activity can be used to treat or prevent a bone disease characterized by a decrease in bone mass relative to a corresponding non-diseased bone by effectuating an increase in bone mass. Such approaches can be used to treat or prevent bone diseases such as osteoporosis, osteopenia, faulty bone formation or resorption, Paget's disease, and bone metastasis. Such methods can be utilized to treat states involving bone fractures and broken bones.

For example, the administration of componds such as soluble peptides, proteins, fusion proteins, or antibodies (including anti-idiotypic antibodies) that bind to and “neutralize” circulating NPY, the natural ligand for the NPY-R, can be used to effectuate an increase in bone mass. Similarly, such compounds as soluble peptides, proteins, fusion proteins, or antibodies (including anti-idiotypic antibodies) can be used to effectuate an increase in bone mass. To this end, peptides corresponding to the ECD of NPY-R, soluble deletion mutants of NPY-R, or either of these NPY-R domains or mutants fused to another polypeptide (e.g., an IgFc polypeptide) can be utilized. Alternatively, anti-idiotypic antibodies or Fab fragments of antiidiotypic antibodies that mimic the NPY-R ECD and neutralize NPY can be used. Alternatively, compounds that inhibit NPY-R homodimerization such that neuropeptide Y's affinity for the neuropeptide Y receptor is decreased, also can be used. Devos et al., 1997, JBC, 272:18304-18310. For treatment, such NPY-R peptides, proteins, fusion proteins, anti-idiotypic antibodies or Fabs are administered to a subject in need of treatment at therapeutically effective levels. For prevention, such NPY-R peptides, proteins, fusion proteins, anti-idiotypic antibodies or Fabs are administered to a subject at risk for a bone disease, for a time and concentration sufficient to prevent the bone disease.

In an alternative embodiment for neutralizing circulating NPY, cells that are genetically engineered to express such soluble or secreted forms of NPY-R may be administered to a patient, whereupon they will serve as “bioreactors” in vivo to provide a continuous supply of the NPY neutralizing protein. Such cells may be obtained from the patient or an MHC compatible donor and can include, but are not limited to, fibroblasts, blood cells (e.g., lymphocytes), adipocytes, muscle cells, endothelial cells etc. The cells are genetically engineered in vitro using recombinant DNA techniques to introduce the coding sequence for the NPY-R ECD, or for NPY-R-Ig fusion protein into the cells, e.g., by transduction (using viral vectors, and preferably vectors that integrate the transgene into the cell genome) or transfection procedures, including, but not limited to, the use of plasmids, cosmids, YACs, electroporation, liposomes, etc. The NPY-R coding sequence can be placed under the control of a strong constitutive or inducible promoter or promoter/enhancer to achieve expression and secretion of the NPY-R peptide or fusion protein. The engineered cells which express and secrete the desired NPY-R product can be introduced into the patient systemically, e.g., in the circulation or intraperitoneally. Alternatively, the cells can be incorporated into a matrix and implanted in the body, e.g., genetically engineered fibroblasts can be implanted as part of a skin graft; genetically engineered endothelial cells can be implanted as part of a vascular graft. (See, for example, Anderson et al. U.S. Pat. No. 5,399,349; and Mulligan & Wilson, U.S. Pat. No. 5,460,959 each of which is incorporated by reference herein in its entirety).

When the cells to be administered are non-autologous cells, they can be administered using well known techniques which prevent the development of a host immune response against the introduced cells. For example, the cells maybe introduced in an encapsulated form which, while allowing for an exchange of components with the immediate extracellular environment, does not allow the introduced cells to be recognized by the host immune system.

In an alternate embodiment, bone disease therapy can be designed to reduce the level of endogenous NPY or NPY-R gene expression, e.g., using antisense or ribozyme approaches to inhibit or prevent translation of NPY or NPY-R mRNA transcripts; triple helix approaches to inhibit transcription of the NPY or NPY-R gene; or targeted homologous recombination to inactivate or “knock out” the NPY or NPY-R gene or its endogenous promoter. Delivery techniques should be preferably designed to cross the blood-brain barrier (see PCT WO89/10134, which is incorporated by reference herein in its entirety). Alternatively, the antisense, ribozyme or DNA constructs described herein could be administered directly to the site containing the target cells.

Antisense approaches involve the design of oligonucleotides (either DNA or RNA) that are complementary to NPY or NPY-R mRNA. The antisense oligonucleotides will bind to the complementary NPY or NPY-R mRNA transcripts and prevent translation. Absolute complementarity, although preferred, is not required. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

The skilled artisan recognizes that modifications of gene expression can be obtained by designing antisense molecules to the control regions of the neuropeptide Y or neuropeptide Y receptor genes, i.e. promoters, enhancers, and introns, as well as to the coding regions of these genes. Such sequences are referred to herein as neuropeptide Y-encoding polynucleotides or neuropeptide Y receptor-encoding polynucleotides, respectively.

Oligonucleotides derived from the transcription initiation site, e.g. between −10 and +10 regions of the leader sequence, are preferred. Oligonucleotides that are complementary to the 5′ end of the message, e.g., the 5′ untranslated sequence up to and including the AUG initiation codon, generally work most efficiently at inhibiting translation. However, sequences complementary to the 3′ untranslated sequences of mRNAs have recently shown to be effective at inhibiting translation of mRNAs as well. See generally, Wagner, R., 1994, Nature 372:333-335. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′-, 3′- or coding region of NPY or NPY-R mRNA, antisense nucleic acids should be at least six nucleotides in length, and are preferably oligonucleotides ranging from 6 to about 50 nucleotides in length. In specific aspects the oligonucleotide is at least nucleotides, at least 17 nucleotides, at least 25 nucleotides or at least 50 nucleotides.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents. (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

While antisense nucleotides complementary to the NPY or NPY-R coding region sequence could be used, those complementary to the transcribed untranslated region are most preferred.

The antisense molecules should be delivered to cells which express the NPY or NPY-R in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

A preferred approach for achieving intracellular concentrations of the antisense sufficient to suppress translation of endogenous mRNAs utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous NPY or NPY-R transcripts and thereby prevent translation of the NPY or NPY-R mRNA, respectively. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), etc. Any type of plasmid, cosmid, YAC or viral vector can be used to prepare the recombinant DNA construct which can be introduced directly into the tissue site. Alternatively, viral, vectors can be used which selectively infect the desired tissue; (e.g., for brain, herpesvirus vectors may be used), in which case administration may be accomplished by another route (e.g., systemically).

Ribozyme molecules-designed to catalytically cleave NPY or NPY-R mRNA transcripts can also be used to prevent translation of NPY or NPY-R mRNA and expression of NPY or NPY-R. While ribozymes that cleave mRNA at site specific recognition sequences can be used to destroy NPY or NPY-R mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591. There are hundreds of potential hammerhead ribozyme cleavage sites within the nucleotide sequence of human NPY and NPY-R cDNA. Preferably, the ribozyme is engineered so that the cleavage recognition site is located near the 5′ end of the NPY or NPY-R mRNA; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena thermophila (known as the IVS, or L-19 WVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent-application No. WO 88/04300 by University Patents Inc.; Been and Cech, 1986, Cell, 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in NPY and NPY-R.

As in the antisense approach, the ribozymes can be composed of modified oligonucleotides (e.g. for improved stability, targeting, etc.) and should be delivered to cells which express NPY and NPY-R in vivo. A preferred method of delivery involves using a DNA construct “encoding” the ribozyme under the control of a strong constitutive pol III or pol II promoter, so that transfected cells will produce sufficient quantities of the ribozyme to destroy endogenous NPY or NPY-R messages and inhibit translation. Because ribozymes, unlike antisense molecules, are catalytic, a lower intracellular concentration is required for efficiency.

Similarly, neuropeptide Y or neuropeptide Y receptor inhibition can be achieved by using “triple helix” base-pairing methodology. Triple helix pairing compromises the ability of the double helix to open sufficiently for the binding of polymerases, transcription factors, or regulatory molecules. Techniques for utilizing triple helix technology are well known to those of skill in the art. See generally Helene (1991) Anticancer Drug Des. 6(6):569-84; Helene (1992) Ann. N.Y. Acad. Sci. 660:27-36; and Maher (1992) Bioassays 14(12):807-15.

Endogenous NPY or NPY-R gene expression can also be reduced by inactivating or “knocking out” the NPY or NPY-R gene or its promoter using targeted homologous recombination. (E.g., see Smithies et al., 1985, Nature 317:230-234; Thomas & Capecchi, 1987, Cell 51:503-512; Thompson et al., 1989 Cell 5:313-321; each of which is incorporated by reference herein in its entirety). For example, a mutant, non-functional NPY or NPY-R (or a completely unrelated DNA sequence) flanked by DNA homologous to the endogenous NPY or NPY-R gene (either the coding regions or regulatory regions) can be used, with or without a selectable marker and/or a negative selectable marker, to transfect cells that express NPY or NPY-R in vivo. Insertion of the DNA construct, via targeted homologous recombination, results in inactivation of the NPY or NPY-R gene. Such approaches are particularly suited in the agricultural field where modifications to ES (embryonic stem) cells can be used to generate animal offspring with an inactive NPY-R (e.g., see Thomas & Capecchi 1987 and Thompson 1989, supra). However, this approach can be adapted for use in humans provided the recombinant DNA constructs are directly administered or targeted to the required site in vivo using appropriate viral vectors, e.g., herpes virus vectors for delivery to brain tissue.

Alternatively, endogenous NPY or NPY-R gene expression can be reduced by targeting deoxyribonucleotide sequences complementary to the regulatory region of the NPY or NPY-R gene (i.e., promoters and/or enhancers) to form triple helical structures that prevent transcription of the NPY or NPY-R gene in target cells in the body. (See generally, Helene, C. 1991, Anticancer Drug Des., 6(6):569-84; Helene, C., et al., 1992, Ann, N.Y. Accad. Sci., 660:27-36; and Maher, L. J., 1992, Bioassays 14(12):807-15).

In yet another embodiment of the invention, the activity of NPY or NPY-R can be reduced using a “dominant negative” approach to effectuate an increase in bone mass. To this end, constructs which encode defective NPY or NPY-Rs can be used in gene therapy approaches to diminish the activity of the NPY or NPY-R in appropriate target cells. For example, nucleotide sequences that direct host cell expression of NPY-Rs in which the CD or a portion of the CD is deleted or mutated can be introduced into target cells (either by in vivo or ex vivo gene therapy methods described above). Alternatively, targeted homologous recombination can be utilized to introduce such deletions or mutations into the subject's endogenous NPY-R gene in the target cells. The engineered cells will express non-functional receptors (i.e., an anchored receptor that is capable of binding its natural ligand, but incapable of signal transduction). Such engineered cells present in the target cells should demonstrate a diminished response to the endogenous NPY ligand, resulting in an increase in bone mass.

An additional embodiment of the present invention is a method to decrease neuropeptide Y levels by increasing breakdown of neuropeptide Y protein, i.e., by binding of an antibody such that the neuropeptide Y protein is targeted for removal. An alternative embodiment of the present invention is a method to decrease neuropeptide Y receptor levels by increasing the breakdown of neuropeptide Y receptor protein, i.e., by binding of an antibody such that the neuropeptide Y receptor protein is targeted for removal. Another embodiment is to decrease neuropeptide Y levels by increasing the synthesis of a soluble form of the neuropeptide Y receptor, which binds to free neuropeptide Y.

Another embodiment of the present invention is a method to administer compounds which affect neuropeptide Y receptor structure, function or homodimerization properties. Such compounds include, but are not limited to, proteins, nucleic acids, carbohydrates or other molecules which upon binding alter neuropeptide Y receptor structure, function, or homodimerization properties, and thereby render the receptor ineffectual in its activity.

5.5.2. Restoration or Increase in NPY or NPY-R Expression or Activity to Decrease Bone Mass

With respect to an increase in the level of normal NPY or NPY-R gene expression and/or gene product activity, NPY or NPY-R nucleic acid sequences can be utilized for the treatment of bone disorders. Where the cause of the disorder is a defective NPY or NPY-R, treatment can be administered, for example, in the form of gene replacement therapy. Specifically, one or more copies of a normal NPY or NPY-R gene or a portion of the NPY or NPY-R gene that directs the production of an NPY or NPY-R gene product exhibiting normal function, may be inserted into the appropriate cells within a patient or animal subject, using vectors which include, but are not limited to, adenovirus, adeno-associated virus, retrovirus and herpes virus vectors, in addition to other particles that introduce DNA into cells, such as liposomes.

Gene replacement therapy techniques involving NPY-R should be capable of delivering NPY-R gene sequences to neural cell types within patients. Thus, the techniques for delivery of the NPY-R gene sequences should be designed to readily cross the blood-brain barrier, which are well known to those of skill in the art (see, e.g., PCT application, publication No. WO89/10134, which is incorporated herein by reference in its entirety), or, alternatively, should involve direct administration of such NPY-R gene sequences to the site of the cells in which the NPY-R gene sequences are to be expressed. Alternatively, targeted homologous recombination can be utilized to correct the defective endogenous NPY or NPY-R gene in the appropriate tissue. In animals, targeted homologous recombination can be used to correct the defect in ES cells in order to generate offspring with a corrected trait.

Additional methods which may be utilized to increase the overall level of NPY or NPY-R gene expression and/or activity include the introduction of appropriate NPY or NPY-R-expressing cells, preferably autologous cells, into a patient at positions and in numbers which are sufficient to ameliorate the symptoms of bone disorders associated with increased bone mass. Such cells may be either recombinant or non-recombinant. Among the cells which can be administered to increase the overall level of NPY or NPY-R gene expression in a patient are normal cells which express the NPY-R gene, or adipocytes, which express the NPY gene. The cells can be administered at the anatomical site of the target cell type, or as part of a tissue graft located at a different site in the body. Such cell-based gene therapy techniques are well known to those skilled in the art, see, e.g., Anderson, et al., U.S. Pat. No. 5,399,349; Mulligan & Wilson, U.S. Pat. No. 5,460,959.

Finally, compounds, identified in the assays described above, that stimulate or enhance the signal transduced by activated NPY-R, e.g., by activating downstream signaling proteins in the NPY-R cascade and thereby by passing the defective NPY-R, can be used to achieve decreased bone mass. The formulation and mode of administration will depend upon the physico-chemical properties of the compound. The administration should include known techniques that allow for a crossing of the blood-brain barrier.

5.5.3. Gene Therapy Approaches to Controlling NPY and NPY-R Activity and Treating or Preventing Bone Disease

The expression of NPY and NPY-R can be controlled in vivo (e.g. at the transcriptional or translational level) using gene therapy approaches to regulate NPY and NPY-R activity and treat bone disorders. Certain approaches are described below.

With respect to an increase in the level of normal NPY and NPY-R gene expression and/or NPY and NPY-R gene product activity, NPY and NPY-R nucleic acid sequences can be utilized for the treatment of bone diseases. Where the cause of the bone disease is a defective NPY or NPY-R gene, treatment can be administered, for example, in the form of gene replacement therapy. Specifically, one or more copies of a normal NPY or NPY-R gene or a portion of the gene that directs the production of a gene product exhibiting normal function, may be inserted into the appropriate cells within a patient or animal subject, using vectors which include, but are not limited to adenovirus, adeno-associated virus, retrovirus and herpes virus vectors, in addition to other particles that introduce DNA into cells, such as liposomes.

Gene replacement therapy techniques should be capable of delivering NPY-R gene sequences to these cell types within patients. Thus, the techniques for delivery of the NPY-R gene sequences should be designed to readily cross the blood-brain barrier, which are well known to those of skill in the art (see, e.g., PCT application, publication No. WO89/10134, which is incorporated herein by reference in its entirety), or, alternatively, should involve direct administration of such NPY-R gene sequences to the site of the cells in which the NPY-R gene sequences are to be expressed.

Alternatively, targeted homologous recombination can be utilized to correct the defective endogenous NPY or NPY-R gene in the appropriate tissue. In animals, targeted homologous recombination can be used to correct the defect in ES cells in order to generate offspring with a corrected trait.

Additional methods which may be utilized to increase the overall level of NPY or NPY-R gene expression and/or activity include the introduction of appropriate NPY or NPY-R-expressing cells, preferably autologous cells, into a patient at positions and in numbers which are sufficient to ameliorate the symptoms of bone disorders, including, but not limited to, osteopetrosis, osteosclerosis and osteochondrosis. Such cells may be either recombinant or non-recombinant. Among the cells which can be administered to increase the overall level of NPY or NPY-R gene expression in a patient are normal cells. The cells can be administered at the anatomical site in the adipose tissue or in the brain, or as part of a tissue graft located at a different site in the body. Such cell-based gene therapy techniques are well known to those skilled in the art, see, e.g., Anderson, et al., U.S. Pat. No. 5,399,349; Mulligan & Wilson, U.S. Pat. No. 5,460,959.

5.6. Pharmaceutical Formulations and Methods of Treating Bone Disorders

The compounds of this invention can be formulated and administered to inhibit a variety of bone disease states by any means that produces contact of the active ingredient with the agent's site of action in the body of a mammal. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

The dosage administered will be a therapeutically effective amount of the compound sufficient to result in amelioration of symptoms of the bone disease and will, of course, vary depending upon known factors such as the pharmacodynamic characteristics of the particular active ingredient and its mode and route of administration; age, sex, health and weight of the recipient; nature and extent of symptoms; kind of concurrent treatment, frequency of treatment and the effect desired.

5.6.1 Dose Determinations

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Specific dosages may also be utilized for antibodies. Typically, the preferred dosage is 0.1 mg/kg to 100 mg/kg of body weight (generally 10 mg/kg to 20 mg/kg), and if the antibody is to act in the brain, a dosage of 50 mg/kg to 100 mg/kg is usually appropriate. If the antibody is partially human or fully human, it generally will have a longer half-life within the human body than other antibodies. Accordingly, lower dosages of partially human and fully human antibodies is often possible. Additional modifications may be used to further stabilize antibodies. For example, lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

A therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

Moreover, treatment of a subject with a therapeutically effective amount of a protein, polypeptide or antibody can include a single treatment or, preferably, can include a series of treatments. In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5 or 6 weeks.

The present invention further encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents depends upon a number of factors known to those or ordinary skill in the art, e.g., a physician. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.

5.6.2 Formulations and Use

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use. In general, water, a suitable oil, saline, aqueous dextrose (glucose), and related sugar solutions and glycols such as propylene glycol or polyethylene glycols are suitable carriers for parenteral solutions. Solutions for parenteral administration contain preferably a water soluble salt of the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly(lactic acid) or ethylenevinylacetate copolymers. In addition to being incorporated, these agents can also be used to trap the compound in microcapsules.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Useful pharmaceutical dosage forms, for administration of the compounds of this invention can be illustrated as follows:

Capsules: Capsules are prepared by filling standard two-piece hard gelatin capsulates each with the desired amount of powdered active ingredient, 175 milligrams of lactose, 24 milligrams of talc and 6 milligrams magnesium stearate.

Soft Gelatin Capsules: A mixture of active ingredient in soybean oil is prepared and injected by means of a positive displacement pump into gelatin to form soft gelatin capsules containing the desired amount of the active ingredient. The capsules are then washed and dried.

Tablets: Tablets are prepared by conventional procedures so that the dosage unit is the desired amount of active ingredient. 0.2 milligrams of colloidal silicon dioxide, 5 milligrams of magnesium stearate, 275 milligrams of microcrystalline cellulose, 11 milligrams of cornstarch and 98.8 milligrams of lactose. Appropriate coatings may be applied to increase palatability or to delay absorption.

Injectable: A parenteral composition suitable for administration by injection is prepared by stirring 1.5% by weight of active ingredients in 10% by volume propylene glycol and water. The solution is made isotonic with sodium chloride and sterilized.

Suspension: An aqueous suspension is prepared for oral administration so that each 5 millimeters contain 100 milligrams of finely divided active ingredient, 200 milligrams of sodium carboxymethyl cellulose, 5 milligrams of sodium benzoate, 1.0 grams of sorbitol solution U.S.P. and 0.025 millimeters of vanillin.

Gene Therapy Administration: Where appropriate, the gene therapy vectors can be formulated into preparations in solid, semisolid, liquid or gaseous forms such as tablets, capsules, powders, granules, ointments, solutions, suppositories, injections, inhalants, and aerosols, in the usual ways for their respective route of administration. Means known in the art can be utilized to prevent release and absorption of the composition until it reaches the target organ or to ensure timed-release of the composition. A pharmaceutically acceptable form should be employed which does not ineffectuate the compositions of the present invention. In pharmaceutical dosage forms, the compositions can be used alone or in appropriate association, as well as in combination, with other pharmaceutically active compounds.

Accordingly, the pharmaceutical composition of the present invention may be delivered via various routes and to various sites in an animal body to achieve a particular effect (see, e.g., Rosenfeld et al. (1991), supra; Rosenfeld et al., Clin. Res., 39(2), 31 1A (1991 a); Jaffe et al., supra; Berkner, supra). One skilled in the art will recognize that although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, peritoneal, subcutaneous, intradermal, as well as topical administration.

The composition of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and animal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the pharmaceutical composition in the particular host.

Accordingly, the present invention also provides a method of transferring a therapeutic gene to a host, which comprises administering the vector of the present invention, preferably as part of a composition, using any of the aforementioned routes of administration or alternative routes known to those skilled in the art and appropriate for a particular application. The “effective amount” of the composition is such as to produce the desired effect in a host which can be monitored using several end-points known to those skilled in the art. Effective gene transfer of a vector to a host cell in accordance with the present invention to a host cell can be monitored in terms of a therapeutic effect (e.g. alleviation of some symptom associated with the particular disease being treated) or, further, by evidence of the transferred gene or expression of the gene within the host (e.g., using the polymerase chain reaction in conjunction with sequencing, Northern or Southern hybridizations, or transcription assays to detect the nucleic acid in host cells, or using immunoblot analysis, antibody-mediated detection, mRNA or protein half-life studies, or particularized assays to detect protein or polypeptide encoded by the transferred nucleic acid, or impacted in level or function due to such transfer).

These methods described herein are by no means all-inclusive, and further methods to suit the specific application will be apparent to the ordinary skilled artisan. Moreover, the effective amount of the compositions can be further approximated through analogy to compounds known to exert the desired effect.

Furthermore, the actual dose and schedule can vary depending on whether the compositions are administered in combination with other pharmaceutical compositions, or depending on interindividual differences in pharmacokinetics, drug disposition, and metabolism. Similarly, amounts can vary in in vitro applications depending on the particular cell line utilized (e.g., based on the number of adenoviral receptors present on the cell surface, or the ability of the particular vector employed for gene transfer to replicate in that cell line). Furthermore, the amount of vector to be added per cell will likely vary with the length and stability of the therapeutic gene inserted in the vector, as well as also the nature of the sequence, and is particularly a parameter which needs to be determined empirically, and can be altered due to factors not inherent to the methods of the present invention (for instance, the cost associated with synthesis). One skilled in the art can easily make any necessary adjustments in accordance with the exigencies of the particular situation.

The following examples are offered by way of example, and are not intended to limit the scope of the invention in any manner.

### 6 EXAMPLES

The results shown herein demonstrate that modulation of NPY signaling can be used to modulate bone mass, and therefore bone disease characterized by increased or decreased bone mass relative to that of corresponding non-diseased bone. The results shown herein demonstrate that direct administration of NPY into the brains of wild type rats causes a decrease in bone volume and bone mass as compared with control, PBS-treated mice.

6.1 Generation, Characterization and Treatment of Animals

Bone specimens were processed as described (Ducy et al., 1999, Genes Dev 13, 1025-1036).

6.2 Histologic and Histomorphometric Analyses.

Histological analyses were performed on undecalcified sections stained with the von Kossa reagent and counterstained with Kernechtrot (Amling et al., 1999, Endocrinology, 140: 4982-4987). Double labeling technique with calcein has been described (Amling et al., 1999, Endocrinology, 140: 4982-4987). Static and dynamic histomorphometric analyses were performed according to standard protocols (Parfitt et al., 1987, J Bone Min Res, 2:595-610) using the Osteomeasure Analysis System (Osteometrics, Atlanta). Statistical differences between groups (n=4 to 6) were assessed by Student's test.

6.3 Intracerebroventricular Infusion.

Animals were anesthetized with avertin and placed on a stereotaxic instrument (Stoelting). The calvaria was exposed and a 0.7 mm hole was drilled upon bregma. A 28-gauge cannula (Brain infusion kit II, Alza) was implanted into the third ventricle according to the following coordinates: midline, −0.3 AP, 3 mm ventral (0 point bregma). The cannula was secure to the skull with cyanoacrylate, and attached with Tygon tubing to an osmotic pump (Alza) placed in the dorsal subcutaneous space of the animal. The rate of delivery was 0.5 μl/hour (75 ng/hr) of NPY (Sigma) or PBS for 28 days.

6.4 Intracerebroventricular Infusion of NPY Reduces Bone Mass in wt Mice

The issue of whether NPY binding to its receptor would affect bone mass was examined in this example. Specifically, PBS or NPY was delivered as discussed above, in Secion 6.3. The pumps were left in place for 28 days and double-labeling with calcein was performed to measure the bone formation parameters.

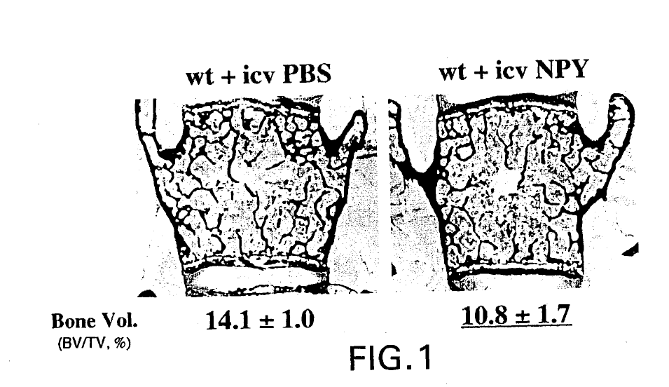

Histological analysis was performed as described above. FIG. 1 shows a decrease in bone volume and bone mass in the NPY-treated mice as compared with the PBS-treated mice.

All patents and publications mentioned in the specifications are indicative of the levels of those skilled in the art to which the invention pertains. All patents and publications are herein incorporated by reference to the same extent as if each individual publication was specifically and individually indicated to be incorporated by reference.

One skilled in the art readily appreciates that the patent invention is well adapted to carry out the objectives and obtain the ends and advantages mentioned as well as those inherent therein. NPY, NPY receptor, NPY antibodies, NPY analogs, NPY antagonists, pharmaceutical compositions, treatments, methods, procedures and techniques described herein are presently representative of the preferred embodiments and are intended to be. exemplary and are not intended as limitations of the scope. Changes therein and other uses will occur to those skilled in the art which are encompassed within the spirit of the invention or defined by the scope of the pending claims.

What is claimed is:

1. A method of treating a bone disease comprising:
administering to a mammal in need of said treatment a therapeutically effective amount of a compound that lowers NPY level in blood serum, wherein the bone disease is characterized by a decreased bone mass relative to that of corresponding non-diseased bone.

2. The method of claim 1, wherein said NPY level is lowered by lowering NPY synthesis.

3. The method of claim 2, wherein said compound is an antisense, ribozyme or triple helix sequence of a NPY-encoding polynucleotide.

4. The method of claim 1, wherein said bone disease is selected from the group consisting of osteoporosis, osteopenia, and Paget's disease.

5. A method of treating a bone disease comprising:
administering to a mammal in need of said treatment a therapeutically effective amount of a compound that lowers NPY level in cerebrospinal fluid, wherein the bone disease is characterized by a decreased bone mass relative to that of corresponding non-diseased bone.

6. The method of claim 5, wherein said compound binds NPY in blood.

7. The method of claim 6, wherein said bone disease is selected from the group consisting of osteoporosis, osteopenia, and Paget's disease.

8. A method of treating a bone disease comprising:
administering to a mammal in need of said treatment a therapeutically effective amount of a compound, wherein the bone disease is characterized by a decreased bone mass relative to that of corresponding non-diseased bone, and wherein the compound is selected from the group consisting of: an antibody which specifically binds NPY, a soluble NPY receptor polypeptide, and derivatives of naphthalenes, benzofuran, benzothiophenes and indoles; raloxifene; 3-(4-methoxyphenyl)-4-[4-(2-pyrrolidin-1-ylethoxy)benzoyl-1,2-dihydronaphthalene, citrate salt; 3-phenyl-4-[4-(2-pyrrolidin-1-ylethoxy)benzoyl]-7-methoxy-1,2-dihydronaphthalene; 3-phenyl-4-[4-(2-pyrrolidin-1-ylethoxy)benzoyl]-1,2-dihydronaphthalene; 1-[4-(2-pyrrolidin-1-ylethoxy)benzoyl]-2-phenylnaphthalene, citrate salt; 3-(4-methoxyphenyl)-4-[4-[2-(piperidin-1-yl)ethoxy]benzoyl]-1,2-dihydronaphthalene, citrate salt; 3-(4-methoxyphenyl)-4-[4-(2-dimethylaminoethoxy)benzoyl]-1,2-dihydronaphthalene, citrate salt; 3-(4-hydroxyphenyl)-4-[4-[2-(pyrrolidin-1-yl)ethoxy]benzoyl]-1,2-dihydronaphthalene, mesylate salt; 3-(4-methoxyphenyl)-4-[4-[2-(hexamethyleneimin-1-yl)benzoyl]-1,2-dihydronaphthalene, mesylate salt; 3-(4-methoxyphenyl)-4-[4-[2-(piperidin-1-yl)ethoxy]benzoyl]-1,2-dihydronaphthalene, mesylate salt; 3-(4-methoxyphenyl)-4-(4-diethylaminoethoxybenzoyl)-1,2-dihydronaphthalene, mesylate salt; 3-(4-methoxyphenyl)-4-(4-diisopropylaminoethoxybenzoyl)-1,2-dihydronaphthalene, mesylate salt; 3-hydroxy-4-[4-[2-(pyrrolidin-1-yl)ethoxy]benzoyl]-1,2-dihydronaphthalene, sodium salt; 2-(4-methoxyphenyl)-1-[4-[2-(pyrrolidin-1-yl)ethoxy]benzoyl]naphthalene, mesylate salt; 3-(4-methoxyphenyl)-4-[4-[2-(piperidin-1-yl)ethoxy]benzoyl]-7-methoxy-1,2-d ihydronaphthalene, mesylate salt; 3-(4-methoxyphenyl)-4-[4-(2-dimethylaminoethoxy)benzoyl]-1,2-dihydronaphthalene, 2-hydroxy-1,2,3-propanetricarboxylic acid salt; 3-(4-methoxyphenyl)-4-[4-[2-(N-methyl-1-pyrrolidinium)ethoxy]benzoyl]-1,2-dihydronaphthalene, iodide salt; and 3-(4-methoxyphenyl)-4-[4-[2-(pyrrolidin-1-yl)ethoxy]benzoyl]-1,2-dihydronaphthalene, mesylate salt.

9. The method of claim 8, wherein said antibody is a monoclonal antibody.

10. The method of claim 8, wherein said antibody is a human or chimeric antibody.

11. The method of claim 10, wherein said antibody is a humanized antibody.

12. The method of claim 8, wherein said bone disease is selected from the group consisting of osteoporosis, osteopenia, and Paget's disease.

13. A method of treating a bone disease comprising:
administering to a mammal in need of said treatment a therapeutically effective amount of a compound that lowers the level of inositol phosphate or extracellular signal-regulated kinase, wherein the bone disease is characterized by a decreased bone mass relative to that of corresponding non-diseased bone.

14. The method of claim 13, wherein said compound is a NPY receptor antagonist.

15. The method of claim 14, wherein said NPY receptor antagonist is selected from the group consisting of: α-alkoxy and α-thioalkoxyamide compositions; dihydropyridine based compounds; substituted benzylamine derivatives; dihydropyrimidone derivatives; naphthimidazolyl derivatives; dimesylate salts); and substituted benzofurans, benzothiophenes or indoles.

16. The method of claim, 14, wherein said NPY receptor antagonist is an antibody selected from the group consisting of an antibody which specifically binds NPY and an antibody which specifically binds NPY receptor.

17. The method of claim 13, wherein said bone disease is selected from the group consisting of osteoporosis, osteopenia, and Paget's disease.

18. The method of claim 1, 5 or 13 further comprising administering to the mammal a therapeutically effective amount of a selective estrogen receptor modulator.

19. The method of claim 18, wherein said selective estrogen receptor modulator is estradiol.

20. A method of preventing a bone disease comprising:
administering to a mammal at risk for the bone disease a compound that lowers NPY level in blood serum, at a concentration sufficient to prevent the bone disease, wherein the bone disease is characterized by a decreased bone mass relative to that of corresponding non-diseased bone.

21. The method of claim 20, wherein said NPY level is lowered by lowering NPY synthesis.

22. The method of claim 21, wherein said compound is an antisense, ribozyme or triple helix sequence of a NPY-encoding polynucleotide.

23. The method of claim 20, wherein said bone disease is selected from the group consisting of osteoporosis, osteopenia, and Paget's disease.

24. A method of preventing a bone disease comprising:
administering to a mammal at risk for the bone disease a compound that lowers NPY level in cerebrospinal fluid, at a concentration sufficient to prevent the bone disease, wherein the bone disease is characterized by a decreased bone mass relative to that of corresponding non-diseased bone.

25. The method of claim 24, wherein said compound binds NPY in blood.

26. The method of claim 24, wherein said bone disease is selected from the group consisting of osteoporosis, osteopenia, and Paget's disease.

27. A method of preventing a bone disease comprising:
administering to a mammal at risk for the bone disease a compound at a concentration sufficient to prevent the bone disease, wherein the bone disease is characterized by a decreased bone mass relative to that of corresponding non-diseased bone, and wherein the compound is selected from the group consisting of: an antibody which specifically binds NPY, a soluble NPY receptor polypeptide, and derivatives of derivatives of naphthalenes, benzofuran, benzothiophenes and indoles; raloxifene; 3-(4-methoxyphenyl)-4-[4-(2-pyrrolidin-1-ylethoxy)benzoyl-1,2-dihydronaphthalene, citrate salt; 3-phenyl-4-[4-(2-pyrrolidin-1-ylethoxy)benzoyl]-7-methoxy-1,2-dihydronaphthalene; 3-phenyl-4-[4-(2-pyrrolidin-1-ylethoxy)benzoyl]-1,2-dihydronaphthalene; 1-[4-(2-pyrrolidin-1-ylethoxy)benzoyl]-2-phenylnaphthalene, citrate salt; 3-(4-methoxyphenyl)-4-[4-[2-(piperidin-1-yl)ethoxy]benzoyl]-1,2-dihydronaphthalene, citrate salt; 3-(4-methoxyphenyl)-4-[4-(2-dimethylaminoethoxy)benzoyl]-1,2-dihydronaphthalene, citrate salt; 3-(4-hydroxyphenyl)-4-[4-[2-(pyrrolidin-1-yl)ethoxy]benzoyl]-1,2-dihydronaphthalene, mesylate salt; 3-(4-methoxyphenyl)-4-[4-[2-(hexamethyleneimin-1-yl)benzoyl]-1,2-dihydronaphthalene, mesylate salt; 3-(4-methoxyphenyl)-4-[4-[2-(piperidin-1-yl)ethoxy]benzoyl]-1,2-dihydronaphthalene, mesylate salt; 3-(4-methoxyphenyl)-4-(4-diethylaminoethoxybenzoyl)-1,2-dihydronaphthalene, mesylate salt; 3-(4-methoxyphenyl)-4-(4-diisopropylaminoethoxybenzoyl)-1,2-dihydronaphthalene, mesylate salt; 3-hydroxy-4-[4-[2-(pyrrolidin-1-yl)ethoxy]benzoyl]-1,2-dihydronaphthalene, sodium salt; 2-(4-methoxyphenyl)-1-[4-[2-(pyrrolidin-1-yl)ethoxy]benzoyl]naphthalene, mesylate salt; 3-(4-methoxyphenyl)-4-[4-[2-(piperidin-1-yl)ethoxy]benzoyl]-7-methoxy-1,2-d ihydronaphthalene, mesylate salt; 3-(4-methoxyphenyl)-4-[4-(2-dimethylaminoethoxy)benzoyl]-1,2-dihydronaphthalene, 2-hydroxy-1,2,3-propanetricarboxylic acid salt; 3-(4-methoxyphenyl)-4-[4-[2-(N-methyl-1-pyrrolidinium)ethoxy]benzoyl]-1,2-dihydronaphthalene, iodide salt; and 3-(4-methoxyphenyl)-4-[4-[2-(pyrrolidin-1-yl)ethoxy]benzoyl]-1,2-dihydronaphthalene, mesylate salt.

28. The method of claim 27, wherein said antibody is a monoclonal antibody.

29. The method of claim 27, wherein said antibody is a human or chimeric antibody.

30. The method of claim 29, wherein said antibody is a humanized antibody.

31. The method of claim 27, wherein said bone disease is selected from the group consisting of osteoporosis, osteopenia, and Paget's disease.

32. A method of preventing a bone disease comprising:
administering to a mammal at risk for the bone disease a compound that lowers the level of inositol phosphate or extracellular signal-regulated kinase, at a concentration sufficient to prevent the bone disease, wherein the bone disease is characterized by a decreased bone mass relative to that of corresponding non-diseased bone.

33. The method of claim 32, wherein said compound is a NPY -receptor antagonist.

34. The method of claim 33, wherein said NPY receptor antagonist is selected from the group consisting of: α-alkoxy and α-thioalkoxyamide compositions; dihydropyridine based compounds; substituted benzylamine derivatives; dihydropyrimidone derivatives; naphthimidazolyl derivatives; dimesylate salts); and substituted benzofurans, benzothiophenes or indoles.

35. The method of claim 33, wherein said NPY receptor antagonist is selected from the group consisting of an antibody which specifically bind NPY and an antibody which specifically binds NPY receptor.

36. The method of claim 32, wherein said bone disease is selected from the group consisting of osteoporosis, osteopenia, and Paget's disease.

37. A method of diagnosing a bone disease in a mammal comprising:
(a) measuring NPY levels in blood serum of a mammal; and
(b) comparing the level measured in (a) to the NPY level in control blood serum,
so that if the level obtained in (a) is higher than that of the control, the mammal is diagnosed as exhibiting the bone disease, wherein the bone disease is characterized by a decreased bone mass relative to that of corresponding non-diseased bone.

38. The method of claim 37, wherein said mammal is a human.

39. The method of claim 37, wherein said bone disease is selected from the group consisting of osteoporosis, osteopenia, and Paget's disease.

40. A method of diagnosing a bone disease in a mammal comprising:
(a) measuring NPY levels in cerebrospinal fluid of a mammal; and
(b) comparing the level measured in (a) to the NPY level in control cerebrospinal fluid,
so that if the level obtained in (a) is higher than that of the control, the mammal is diagnosed as exhibiting the bone disease, wherein the bone disease is characterized by a decreased bone mass relative to that of corresponding non-diseased bone.

41. The method of claim 40, wherein said mammal is a human.

42. The method of claim 40, wherein said bone disease is selected from the group consisting of osteoporosis, osteopenia, and Paget's disease.

43. A method for identifying a compound to be tested for an ability to modulate bone mass in a mammal, comprising:
(a) contacting a test compound with a polypeptide; and
(b) determining whether the test compound binds the polypeptide, so that if the test compound binds the polypeptide, then a compound to be tested for an ability to modulate bone mass is identified,
wherein the polypeptide is selected from the group consisting of a NPY polypeptide and a NPY receptor polypeptide.

44. The method of claim 43, wherein said polypeptide is a human polypeptide

45. The method of claim 43, wherein said ability to modulate bone mass is the ability to increase bone mass.

46. The method of claim 43, wherein said ability to modulate bone mass is the ability to decrease bone mass.

47. A method for identifying a compound that modulates bone mass in a mammal, comprising:
(a) contacting test compounds with a polypeptide;
(b) identifying a test compound that binds the polypeptide; and
(c) administering the test compound in (b) to a non-human mammal, and determining whether the test compound modulates bone mass in the mammal relative to that of a corresponding bone in an untreated control non-human mammal,
wherein the polypeptide is selected from the group consisting of a NPY polypeptide and a NPY receptor polypeptide, so that if the test compound modulates bone mass, then a compound that modulates bone mass in a mammal is identified.

48. The method of claim 47, wherein said polypeptide is a human polypeptide.

49. The method of claim 47, wherein said ability to modulate bone mass is the ability to increase bone mass.

50. The method of claim 47, wherein said ability to modulate bone mass is the ability to decrease bone mass.

51. A method for identifying a compound to be tested for an ability to modulate bone mass in a mammal, comprising:
(a) contacting a test compound with a NPY polypeptide and a NPY receptor polypeptide for a time sufficient to form NPY/NPY receptor complexes; and
(b) measuring NPY/NPY receptor complex level, so that if the level measured differs from that measured in the absence of the test compound, then a compound to be tested for an ability to modulate bone mass is identified.

52. The method of claim 51, wherein said NPY polypeptide is a human polypeptide.

53. The method of claim 51, wherein said NPY receptor polypeptide is a human polypeptide.

54. The method of claim 51, wherein said ability to modulate bone mass is the ability to increase bone mass.

55. The method of claim 51, wherein said ability to modulate bone mass is the ability to decrease bone mass.

56. A method for identifying a compound to be tested for an ability to decrease bone mass in a mammal, comprising:
(a) contacting a test compound with a cell which expresses a functional NPY receptor; and
(b) determining whether the test compound activates the NPY receptor, wherein if the compound activates the NPY receptor a compound to be tested for an ability to decrease bone mass in a mammal is identified.

57. A method for identifying a compound that decreases bone mass in a mammal, comprising:
(a) contacting a test compound with a cell that expresses a functional NPY receptor, and determining whether the test compound activates the NPY receptor;
(b) administering a test compound identified in (a) as activating the NPY receptor to a non-human animal, and determining whether the test compound decreases bone mass of the animal relative to that of a corresponding bone of a control non-human animal,
so that if the test compound decreases bone mass, then a compound that decreases bone mass in a mammal is identified.

58. A method for identifying a compound to be tested for an ability to increase bone mass in a mammal, comprising:
(a) contacting a NPY polypeptide and a test compound with a cell that expresses a functional NPY receptor; and
(b) determining whether the test compound lowers activation of the NPY receptor relative to that observed in the absence of the test compound;
wherein a test compounds that lowers activation of the NPY receptor is identified as a compound to be tested for an ability to increase bone mass in a mammal.

59. A method for identifying a compound that increases bone mass in a mammal, comprising:
(a) contacting a NPY polypeptide and a test compound with a cell that expresses a functional NPY receptor, and determining whether the test compound decreases activation of the NPY receptor;
(b) administering a test compound identified in (a) as decreasing NPY receptor to a non-human animal, and determining whether the test compound increases bone mass of the animal relative to that of a corresponding bone of a control non-human animal,
so that if the test compound increases bone mass, then a compound that increases bone mass in a mammal is identified.

60. The method of claim 56, 57, 58 or 59 in which activation of the NPY receptor is determined by measuring levels of inositol phosphate or extracellular signal-regulated kinase.
